(19) United States
(12) Patent Application Publication
(10) Pub. No.: US 2010/0022626 A1
(43) Pub. Date: Jan. 28, 2010

(54) Modulators of cell migration and methods of identifying same

(76) Inventors: Benjamin Geiger, Rechovot (IL); Zvi Kam, Tel-Aviv (IL); Suha Naffar-Abu-Amara, Ramla (IL)

Correspondence Address: MARTIN D. MOYNIHAN d/b/a PRTSI, INC., P.O. BOX 16446, ARLINGTON, VA, 22215, US

(21) Appl. No.: 11/991,165

(22) PCT Filed: Aug. 29, 2006
(86) PCT No.: PCT/IL06/01000

§ 371 Date: Aug. 20, 2009

Related U.S. Application Data
(60) Provisional application No. 60/711,673, filed on Aug. 29, 2005.

(51) Int. Cl. A61K 31/7052; C12N 5/00; A61P 35/00

(52) U.S. Cl. 514/44 R; 435/375

(57) ABSTRACT

The present invention relates to a method of measuring cell migration, the method comprising (a) contacting a cell with a plurality of polystyrene non-fluorescent beads so as to generate a migratory track; and (b) analyzing at least one morphometric parameter of said migratory track, the morphomotric parameter being indicative of cell migration. The present invention also relates to methods of treating a medical condition associated with cell migration, the method comprising administering to a subject in need thereof a therapeutically effective amount of an agent capable of modulating the activity or expression of genes identified using the above assay, thereby treating the medical condition associated with cell migration.

### FIELD AND BACKGROUND OF THE INVENTION

The present invention relates to modulators of cell migration and methods of identifying same.

Cell migration plays a central role in a wide variety of biological phenomena including embryonic development, angiogenesis, wound healing, immune response, and inflammation. In embryogenesis, cellular migrations are a recurring theme in important morphogenic processes ranging from gastrulation to development of the nervous system. In the adult organism, cell migration remains prominent in both physiological and pathological conditions. Migration of fibroblasts and vascular endothelial cells is essential for wound healing. In metastasis, tumor cells migrate from the initial tumor mass throughout the whole body. Directed tumor cell motility by chemotaxis is the final step of tumor invasion, and the modulation, e.g., inhibition of this process has been a major focus of research.

Cell migration is also central to the immune response. Lymphocytes play a number of crucial roles in immune responses, including direct killing of virus-infected cells, cytokine and antibody production, and facilitation of B cell responses. Lymphocytes are also involved in acute and chronic inflammatory disease; asthma; allergies; autoimmune diseases such as scleroderma, pernicious anemia, multiple sclerosis, myasthenia gravis, IDDM, rheumatoid arthritis, systemic lupus erythematosus, and Crohn's disease; and organ and tissue transplant disease, e.g., graft vs. host disease.

Migratory processes are highly complex cellular activities that require cell polarization, well coordinated changes in the actin and microtubule cytoskeleton, production of membrane protrusions, and spatiotemporally-controlled formation and turn-over of cell-matrix adhesions. Different cell types move with different velocities, persistence and/or directionality, in response to external and internal cues.

In view of the significant involvement of cell migration in such basic physiological processes as well as in pathological states, a growing need for experimental systems that enable quantification of migratory parameters is becoming increasingly apparent.

Migratory assays are primarily instrumental in identifying migration-related genes, as well as for the discovery and development of specific pharmaceutical agents that modulate the motility of a variety of target cells. For these and other purposes, a migratory assay should bear relevance to the in vivo context, should be compatible with high-throughput screening approach, and should provide detailed, quantitative information about as many migratory features as possible. Ideally, measurement of such features should include the dynamic properties associated with cellular navigation.

Successful retrieval of such “multidimensional information” depends upon the particular migration assay used. Naturally, live cell data based on time-lapse movies can be most informative for the quantification of dynamic events in individual cells or cell populations (Dai et al., 2005, Exp Cell Res 311, 272-80). Yet this approach is incompatible with high-throughput analysis. Another common migration assay; namely, the trans-well migration system (Mastyugin et al., 2004, J Biomol Screen 9, 712-8), provides general information about the migratory potential of the cell population at large, but fails to reveal specific motile features of individual cells. Similar limitations are also inherent in another approach to the quantification of cell migration, namely the in vitro “wound closure” assay, in which the extent of cell migration into a wound introduced into a confluent cell culture is measured (Yarrow et al., 2004, BMC Biotechnol 4, 21).

Other common approaches for quantifying migratory parameters recording of the migration “history” are based on such assays as phagokinetic track (PKT) formation on flat surfaces (Albrecht-Buehler, 1977, Cell 11, 395-404; Kawa et al., 1997, FEBS Lett 420, 196-200; Lin et al., 2005, Mol Cancer 4, 21; Scott et al., 2000, Anal Biochem 287, 343-4; Zetter, 1980, Nature 285, 41-3).

The phagokinetic track assay has been used for studying the migratory patterns of various cell types, matrix remodeling, and perturbation of cell migration by chemical or genetic modulators (Baudoux et al., 2000, Eur J Cell Biol 79, 41-51; Ohmori et al., 2001, J Biol Chem 276, 5274-80; Onishi et al., 2003, Clin Exp Metastasis 20, 51-8; Takanami et al., 2002, Oncol Rep 9, 125-8). Such studies are of particular relevance to cancer cell motility, which is believed to reflect the invasive or metastatic potential of these cells in vivo. Thus, identification of chemicals that alter cell migration, or specific genes whose perturbation affects cell migration, could potentially be used for the modulation of metastatic cell migration.

However, attempts to upscale the original procedure to a multi-well format for the automated recording of the PKT have thus far been unsuccessful, since the variability between wells, as well as among fields within the same well, was too great, and essentially incompatible with accurate, automatic track segmentation.

A commercial fluorescent micro-bead PKT assay is available [Cell Motility BioApplication and HitKit®; Cellomics Inc., Pittsburg, Pa.]. However, the use of fluorescent microbeads has a number of disadvantages. First, fluorescent beads require the use of life time movies due to the exposure of the cells to florescent light. Second, in order to recognize the cells and thereby analyze the tracks (e.g. analyze the positioning of the cell in the track or determine how many cells are responsible for a particular track), it is necessary to stain them with agents such as phalloidin. However, cell staining may prove difficult and data analysis would require the development of morphometric software for obtaining multiple parameters, which was not mentioned or described.

There is thus a widely recognized need for, and it would be highly advantageous to have, high-throughput screening assays for use in identifying modulators of cell migration devoid of the above limitations

### SUMMARY OF THE INVENTION

According to one aspect of the present invention there is provided a method of treating a medical condition associated with cell migration, the method comprising administering to a subject in need thereof a therapeutically effective amount of an agent capable of modulating the activity or expression of RPL14, thereby treating the medical condition associated with cell migration.

According to further features in preferred embodiments of the invention described below, the modulating is upregulating the activity or expression of RPL14 and whereas the medical condition comprises a tissue damage.

According to still further features in preferred embodiments of the invention described below, the modulating is downregulating the activity or expression of RPL14 and whereas the medical condition is selected from the group consisting of cancer and cancer metastasis.

According to still further features in preferred embodiments of the invention described below, the agent is an isolated polynucleotide which comprises a nucleic acid sequence encoding an RPL14 polypeptide.

According to another aspect of the present invention there is provided a method of treating a medical condition associated with cell migration, the method comprising administering to a subject in need thereof a therapeutically effective amount of an agent capable of modulating the activity or expression of ZFL36L1, thereby treating the medical condition associated with cell migration.

According to further features in preferred embodiments of the invention described below, the modulating is upregulating the activity or expression of ZFL36L1 and whereas the medical condition comprises a tissue damage.

According to still further features in preferred embodiments of the invention described below, the modulating is downregulating the activity or expression of ZFL36L1 and whereas the medical condition is selected from the group consisting of cancer and cancer metastasis.

According to still further features in preferred embodiments of the invention described below, the agent is an isolated polynucleotide which comprises a nucleic acid sequence encoding a ZFL36L1 polypeptide.

According to yet another aspect of the present invention there is provided a method of treating a medical condition associated with cell migration, the method comprising administering to a subject in need thereof a therapeutically effective amount of an agent capable of modulating the activity or expression of CHP, thereby treating the medical condition associated with cell migration.

According to further features in preferred embodiments of the invention described below, the modulating is upregulating the activity or expression of CHP and whereas the medical condition comprises a tissue damage.

According to still further features in preferred embodiments of the invention described below, the modulating is downregulating the activity or expression of CHP and whereas the medical condition is selected from the group consisting of cancer and cancer metastasis.

According to still further features in preferred embodiments of the invention described below, the agent is an isolated polynucleotide which comprises a nucleic acid sequence encoding a CHP polypeptide.

According to yet another aspect of the present invention there is provided a method of treating a medical condition associated with cell migration, the method comprising administering to a subject in need thereof a therapeutically effective amount of an agent capable of modulating the activity or expression of MFGE8, thereby treating the medical condition associated with cell migration.

According to further features in preferred embodiments of the invention described below, the modulating is upregulating the activity or expression of MFGE8 and whereas the medical condition comprises a tissue damage.

According to still further features in preferred embodiments of the invention described below, the modulating is downregulating the activity or expression of MFGE8 and whereas the medical condition is selected from the group consisting of cancer and cancer metastasis.

According to still further features in preferred embodiments of the invention described below, the agent is an isolated polynucleotide which comprises a nucleic acid sequence encoding a MFGE8 polypeptide.

According to still further features in preferred embodiments of the invention described below, the agent is a MFGE8 polypeptide.

According to yet another aspect of the present invention there is provided a method of treating a medical condition associated with cell migration, the method comprising administering to a subject in need thereof a therapeutically effective amount of an agent capable of modulating the activity or expression of Homo Sapiens chromosome 1 clone RP4-7, thereby treating the medical condition associated with cell migration.

According to further features in preferred embodiments of the invention described below, the modulating is upregulating the activity or expression of Homo Sapiens chromosome 1 clone RP4-7 and whereas the medical condition comprises a tissue damage.

According to still further features in preferred embodiments of the invention described below, the modulating is downregulating the activity or expression of Homo Sapiens chromosome 1 clone RP4-7 and whereas the medical condition is selected from the group consisting of cancer and cancer metastasis.

According to still further features in preferred embodiments of the invention described below, the agent is an isolated polynucleotide which comprises a nucleic acid sequence encoding a Homo Sapiens chromosome 1 clone RP4-7 polypeptide.

According to yet another aspect of the present invention there is provided a method of treating tissue damage, the method comprising administering to a subject in need thereof a therapeutically effective amount of an agent capable of upregulating the activity or expression of BIRC5, thereby treating the tissue damage.

According to still further features in preferred embodiments of the invention described below, the agent is an isolated polynucleotide which comprises a nucleic acid sequence encoding a BIRC5 polypeptide.

According to yet another aspect of the present invention there is provided a method of treating a cancer metastasis, the method comprising administering to a subject in need thereof a therapeutically effective amount of an agent capable of downregulating the activity or expression of BIRC5, thereby treating the cancer metastasis.

According to yet another aspect of the present invention there is provided a method of treating tissue damage, the method comprising administering to a subject in need thereof a therapeutically effective amount of an agent capable of upregulating the activity or expression of EEF1 gamma, thereby treating the tissue damage.

According to still further features in preferred embodiments of the invention described below, the agent is an isolated polynucleotide which comprises a nucleic acid sequence encoding a EEF1 gamma polypeptide.

According to yet another aspect of the present invention there is provided a method of treating a cancer metastasis, the method comprising administering to a subject in need thereof a therapeutically effective amount of an agent capable of downregulating the activity or expression of EEF1 gamma, thereby treating the cancer metastasis.

According to yet another aspect of the present invention there is provided a method of treating tissue damage, the method comprising administering to a subject in need thereof a therapeutically effective amount of an agent capable of upregulating the activity or expression of HOXB7, thereby treating the tissue damage.

According to still further features in preferred embodiments of the invention described below, the agent is an isolated polynucleotide which comprises a nucleic acid sequence encoding a HOXB7 polypeptide.

According to yet another aspect of the present invention there is provided a method of treating a cancer metastasis, the method comprising administering to a subject in need thereof a therapeutically effective amount of an agent capable of downregulating the activity or expression of HOXB7, thereby treating the cancer metastasis.

According to yet another aspect of the present invention there is provided a method of treating tissue damage, the method comprising administering to a subject in need thereof a therapeutically effective amount of an agent capable of up-regulating the activity or expression of DR-nm23, thereby treating the tissue damage.

According to still further features in preferred embodiments of the invention described below, the agent is an isolated polynucleotide which comprises a nucleic acid sequence encoding a DR-nm23 polypeptide.

According to yet another aspect of the present invention there is provided a method of treating cancer or cancer metastasis, the method comprising administering to a subject in need thereof a therapeutically effective amount of an agent capable of down-regulating the activity or expression of DR-nm23, thereby treating cancer or cancer metastasis.

According to further features in preferred embodiments of the invention described below, the tissue damage is selected from the group consisting of a wound, a spinal cord injury, brain injury, brain trauma and a neuronal disease or disorder.

According to still further features in preferred embodiments of the invention described below, the modulating is effected at the nucleic acid level.

According to still further features in preferred embodiments of the invention described below, the agent is selected from the group consisting of an antisense, an siRNA, a ribozyme and a DNAzyme.

According to still further features in preferred embodiments of the invention described below, the modulating is effected at the protein level.

According to still further features in preferred embodiments of the invention described below, the agent is an antibody.

According to still further features in preferred embodiments of the invention described below, the modulating is effected in vivo.

According to still further features in preferred embodiments of the invention described below, the modulating is effected ex vivo.

According to a further aspect of the present invention, there is provided a method of measuring cell migration, the method comprising: (a) contacting a cell with a plurality of polystyrene non-fluorescent beads so as to generate a migratory track; and (b) analyzing at least one morphometric parameter of the migratory track, the morphomotric parameter being indicative of cell migration.

According to further features in preferred embodiments of the invention described below, the at least one morphometric parameter is selected from the group consisting of length, persistence, velocity, lamellar activity and directionality.

According to further features in preferred embodiments of the invention described below, the cells are not stained.

According to further features in preferred embodiments of the invention described below, the polystyrene beads are between about 340-400 nm in diameter.

According to further features in preferred embodiments of the invention described below, the polystyrene beads comprise carboxyl groups on their surface.

According to further features in preferred embodiments of the invention described below, a density of the carboxyl groups is between 90-185 μEq/g.

According to still further features in preferred embodiments of the invention described below, the density of the carboxyl groups is between 160-185 μEq/g.

According to still a further aspect of the present invention there is provided a method of screening for a cell migration affecting agent, the method comprising: (a) treating a cell with a pharmaceutical agent; (b) contacting the cell with a plurality of polystyrene non-fluorescent beads so as to generate a migratory track; and (c) analyzing at least one morphometric parameter of the migratory track wherein a change of the at least one morphometric parameter of the migratory track as compared to a non-treated cell is indicative of a migration affecting agent.

According to still further features in preferred embodiments of the invention described below, the cells are migratory.

According to still further features in preferred embodiments of the invention described below, the cells are non-migratory.

The present invention successfully addresses the shortcomings of the presently known configurations by providing novel modulators of cell migration and methods of identifying same.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic outline of the 96-well plate preparation for the PKT assay.

FIGS. 2A-H are image segmentations defining individual phagokinetic tracks.

FIG. 2A is an original montage of 4×4 fields (1024×1024 pixels) acquired using a 10×/0.4 objective. FIG. 2B is a single field (512×512 pixels, binned 2×2). FIG. 2C is a histogram of pixel intensity: the gray intensity histogram peak (black star) corresponds to background pixels. The small peak of bright intensity (blue star) is contributed by track pixels. Dark pixels, representing cell bodies and some debris (red star), are too few to create a visible histogram peak. The red lines mark the two thresholds separating the background pixels from the cells and tracks. FIGS. 2D-E are images after applying thresholds. Pixels with intensities below the lower threshold (cells and debris) are colored white in FIG. 2D, and pixels with intensity above the higher threshold (tracks) are colored white in FIG. 2E. FIG. 2F is an image where all connected components (objects) of the entire montage image are outlined: Tracks are outlined in red. Objects either too small or too large in area to be included in the image analyses, or located on the borders of the imaged fields, are outlined in blue. FIG. 2G is an enlargement of a segmented field following the binary segmentation. FIG. 2H is the same area as shown in FIG. 2G following multi-scale segmentation, including the axes presentation.

FIGS. 3A-D are schematic depictions of image acquisitions and displays. FIG. 3A is a template of a 96-well plate. FIG. 3B illustrates the positions of 52 fields of which can be acquired within one well. FIG. 3C is a montage of 4×4 images, representing the central area of the well. FIG. 3D is a full-resolution image of one field within the montage. Magnification: 10×. Scale bar: 250 μm.

FIGS. 4A-D are images of phagokinetic tracks with distinct characteristics produced by various cell types. FIG. 4A is a montage of MCF7 cells with a full-resolution single image (inset) superimposed thereupon. FIG. 4B is a montage of MDA-MB-231 cells with a full-resolution single image (inset) superimposed thereupon. FIG. 4C is a montage of B16-F10 cells with a full-resolution single image (inset) superimposed thereupon. FIG. 4D is a montage of H1299 cells with a full-resolution single image (inset) superimposed thereupon. Note the differences in track area and shape, depending on the cell line. Scale bars: 250 μm.

FIGS. 5A-J are images illustrating the morphometry of PKTs formed by H1299, B16-F10, MCF7 and MDA-MB-231 cells. FIG. 5A are original images. FIG. 5B illustrate track area (μm²) cleared of beads by the migrating cells (Red). FIG. 5C illustrates minor axes (μm), of the best-fit ellipse and FIG. 5D illustrates major axes (μm), of the best-fit ellipse. FIG. 5E illustrates the axes ratio and FIG. 5F illustrates the perimeter of the track. FIG. 5G illustrates the roughness [Perimeter²/(4π*Area)] of the track. FIG. 5H illustrates the solidity (track area/area of the convex hull (Red+Blue) enclosing the track) of the track. FIG. 5I illustrates the effective velocity (end-to-end distance (red)/migration time) of the cells. FIG. 5J illustrates the total migration speed (length of skeleton and branches (green+blue)/time) of the cells.

FIGS. 6A-E are images illustrating the effects of cytoskeleton-disrupting drugs on H1299 cell migration. FIG. 6A is a montage image of control H1299 cells, the cells exhibit long migratory paths with high persistence. FIG. 6B is a montage image of H1299 cells treated with Latrunculin A (4 μM). FIG. 6C is a montage image of H1299 cells treated with Nocadazole (2.5 μM). Treated wells indicate an inhibited cell motility. FIG. 6D is a montage image of H1299 cells treated with PMA (100 ng/ml). Treated well image shows increase in cell motility. FIG. 6E is a schematic representation of half of a 96-well plate used for the experiment. Wells A1-B6 are control wells, containing H1299 cells with full culture medium, supplemented with 10% FCS. Wells C1-D6 were treated with Latrunculin A. Wells E1-F6 were treated with Nocadazole. Wells G1-H6 were treated with PMA. The color code indicates mean track area, ranging from 5000 μm² (dark blue) to 16,000 μm² (red). Scale bars: 250 μm.

FIGS. 7A-S are montage images illustrating that BIRC5, MFGE8, HOXB7, PKCzeta, ERBB3, SCYB6, CHP, FGF7 and Rho GDI alpha induce MCF7 migration. FIG. 7A is an image of the PKT of GFP-MCF7 control cells, the cells restrain their stationary state. FIGS. 7B and 7C are full resolution single images of MCF7 cells expressing GFP. FIGS. 7D and 7E are full resolution single images of MCF7 cells expressing PKCzeta. FIGS. 7F and 7G are full resolution single images of MCF7 cells expressing Rho GDI. FIGS. 7H and 7I are full resolution single images of MCF7 cells expressing BIRC5. FIGS. 7J and 7K are full resolution single images of MCF7 cells expressing CHP. FIGS. 7L and 7M are full resolution single images of MCF7 cells expressing ERBB3. FIGS. 7N and 7O are full resolution single images of MCF7 cells expressing HOXB7. FIGS. 7P and 7Q are full resolution single images of MCF7 cells expressing SYCB6. FIGS. 7R and 7S are full resolution single images of MCF7 cells expressing MFGE8. FIG. 7T is a full resolution single image of MCF7 cells expressing FGF7. The candidates exhibited an increased migratory phynotype in comparison to the control. They vary in the range of the migration capacity between each other. Scale bars: 250 μm.

FIG. 8 is a bar graph illustrating the difference in migration characteristics between each morphometric parameters of each candidate in comparison to the control among the 80-percentile population. Ratio value of 1 means no difference between the candidate and the control cells among the 80-percentile cell population in the indicated parameter (GFP-control is always 1). Higher or lower numbers than 1 indicate on the increase of decrease folds, respectively, in the tested parameters.

FIGS. 9A-C are auto-correlation test between the PKT morphometric parameters of GFP— control (FIG. 9A), FGF7 and HOXB7 (FIG. 9B) and PKCzeta and ERBB3 (FIG. 9C). Positive numbers means positive correlation between the two correlated parameters, negative numbers means negative correlation. Zero means no correlation and 1 indicate on absolute correlation. Each rectangle divided by white line into two triangles, each triangle shows the correlation test of different candidate. P-value of each correlation results is indicated beneath the correlation score number.

FIGS. 10A-G are montage images illustrating that RPL14, EEF1 gamma, E5, DR-nm23 and ZFL36L1 induce MCF7-ER migration. FIG. 10A is an image of the PKT of GFP-MCF7 control cells. The cells restrain their stationary state. FIG. 10B is a full resolution single image of MCF7 cells expressing GFP. FIG. 10C is a full resolution single image of MCF7 cells expressing RPL14. FIG. 10D is a full resolution single image of MCF7 cells expressing EEF1 gamma. FIG. 10E is a full resolution single image of MCF7 cells expressing E5. FIG. 10F is a full resolution single image of MCF7 cells expressing ZFL36L1. FIG. 10G is a full resolution single image of MCF7 cells expressing DR-nm23. The candidates exhibited an increased migratory phynotype in comparison to the control. They vary in the range of the migration capacity between each other. Scale bars: 250 μm.

### DESCRIPTION OF THE PREFERRED EMBODIMENTS

The present invention is of novel modulators of cell migration and methods of identifying same. Specifically, the present invention is of polypeptides and polynucleotides which may be used to treat medical conditions associated with cell migration.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cell migration plays a central role in a wide variety of biological phenomena including embryonic development, angiogenesis, wound healing, immunity and inflammation. In the adult organism, cell migration remains prominent in both physiological and pathological conditions. For example, migration of fibroblasts and vascular endothelial cells is essential for wound healing. In metastasis, tumor cells migrate from the initial tumor mass throughout the whole body. Directed tumor cell motility by chemotaxis is the final step of tumor invasion, and the modulation of this process has been a major focus of research.

In view of the significant involvement of cell migration in such basic physiological processes as well as in pathological states, a growing need for experimental systems that enable quantification of migratory parameters is becoming increasingly apparent.

The phagokinetic track (PKT) assay has been used for studying the migratory patterns of various cell types, matrix remodeling, and perturbation of cell migration by chemical or genetic modulators. In the original PKT assay, cells were seeded on gold particle-coated cover slips on which they migrated, pushing the gold particles aside or engulfing them as they moved, thereby producing a permanent record of their migratory paths (Albrecht-Buehler, 1977, Cell 12, 333-9). However, attempts to upscale the original procedure to a multi-well format for the automated recording of the PKT have thus far been unsuccessful, since the variability between wells, as well as among fields within the same well, was too great, and essentially incompatible with accurate, automatic track segmentation.

A commercial fluorescent micro-bead PKT assay is available [Cell Motility BioApplication and HitKit®; Cellomics Inc., Pittsburg, Pa.]. The use of fluorescent microbeads has a number of disadvantages over the non-fluorescent polystyrene beads of the present invention. First, fluorescent beads require the use of life time movies due to the exposure of the cells to florescent light. Second, in order to recognize the cells and thereby analyze the tracks, it is necessary to stain them with agents such as phalloidin. A further disadvantage of fluorescent microbeads is that they are subject to decay and therefore have a limited lifetime.

Whilst reducing the present invention to practice, the present inventors have uncovered that the use of non-fluorescent polystyrene beads instead of fluorescent beads can be advantageously used for performing the PKT assay since the assay of the present invention may be accompanied by a very powerful image analysis program a variety, multiple morphometric parameters may be analyzed (e.g. migration persistence, membrane protrusions). Therefore, unlike other conventional methods, dynamic changes may be identified.

In addition, the assay of the present invention may be used to identify agents (e.g. genes) that not only affect migration, but also affect cell adhesion.

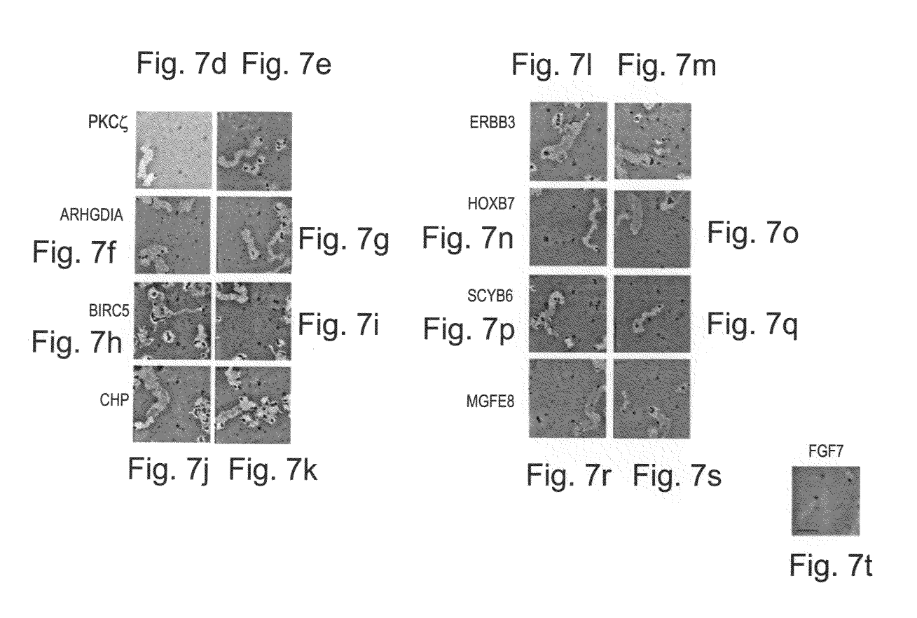

Using the migration assay of the present invention, the present inventors have screened low migratory cells over-expressing genes from two breast cancer cDNA libraries, in order to identify migration inducing genes (Example 3, FIGS. 7A-F and 10A-G).

Using such an approach, a number of pro-migratory genes were uncovered, which, until presently have not been known to be associated with migration.

Thus, according to one aspect of the present invention, there is provided a method of measuring cell migration, the method comprising (a) contacting a cell with a plurality of polystyrene non-fluorescent beads so as to generate a migratory track; and (b) analyzing at least one morphometric parameter of the migratory track, the morphomotric parameter being indicative of cell migration.

As used herein, the phrase “cell migration” refers to the directed movement of cells from one location to another. Any cell type may be analyzed for its ability to migrate using the method of the present invention e.g. prokaryotic cells such as bacterial cells or eukaryotic cells such as mammalian cells. The cells may be inherently capable of migration or may only be capable of migration following induction with a pro-migratory agent or factor.

According to this aspect of the present invention, cells are cultured on beads made of materials such as polystyrene and polymers with similar chemistry which have been previously attached to a suitable surface (e.g. a cell culture dish) so as to form a monolayer. Through meticulous experimentation, the present inventors have shown that both bead dimension and bead surface chemistry greatly and surprisingly affect the beads effectiveness at measuring cell migration. For example, these parameters were shown to effect the capacity of the beads to form a uniform monolayer, to firmly attach to the surface of the well and yet to be effectively cleared by migrating cells.

Accordingly, the present inventors have shown that polystyrene beads between about 300 and 450 nm and even more preferably between about 340-400 nm in diameter are preferably used for this method of the present invention. Preferably, the polystyrene beads comprise carboxyl groups on their surface, at a density preferably between 90-185 μEq/g and even more preferably between 160-185 μEq/g.

According to this aspect of the present invention, the beads are non-fluorescent.

An exemplary bead that was found to be suitable for most cell types according to this aspect of the present were 340 nm diameter, surfactant-free Carboxylated Modified Latex (CML) white polystyrene beads, negatively charged due to carboxylate groups on their surface, with a charge content of 184.7 μEq/g. These beads were shown to form a homogenous and visible monolayer; moreover, their attachment to the substrate was firm enough to prevent spontaneous detachment, yet still weak enough to be removed by migrating cells. Such beads are commercially available e.g. Interfacial Dynamics Corporation-Molecular Probes Microspheres Technologies, USA

As mentioned above, the beads of the present invention are typically pre-attached to a surface. The surface may be pretreated with an agent to aid in the adhesion of the beads. For example, the surface may be treated for a length of time (e.g. two hours) with a fibronectin solution as described in Example 1 hereinbelow. Another agent which may be used to coat the surface is positively charged poly-1-lysine.

Preferably, the number of cells contacted with the polystyrene beads of the present invention is calibrated so as to maximize the number of single cell tracks, yet minimize the number of intersecting tracks. For example, for MCF7 cells, ˜400 cells/well of a 96 well plate was found to be optimal; for B16-F10 cells, 300 cells/well of a 96 well plate was found to be optimal; and for the more motile MDA-MB-231 and H1299 cells, ˜200 cells/well of a 96 well plate was found to be optimal.

Contacting cells with the polystyrene beads of the present invention is effected for a sufficient length of time such that the cells are able to migrate a measurable distance, phagocytosing beads that are in their path (e.g. 5-10 hours). The cells are contacted with the beads in a medium which is at least capable of supporting the migratory activity of the cells.

Following generation of migratory tracks, the cells and beads are preferably fixed. An exemplary fixative is 3% PFA.

Analyzing morphometric parameters of the generated migratory tracks is typically effected using a light microscope. Migrating cells may be distinguished by the dark color around their nucleus (due to the phagocytosis of the beads). Accordingly, the cells do not have to be stained in order to be detected.

When performing large-scale screening, in the step of screening a manual procedure can be followed, although automated screening using robots, such as multiwell attachment for the DeltaVision microscope, Cellomics automated microscope, are preferred. In addition, it is preferable that an image processor such as that described in the Examples section below is also used so that many morphometric parameters may be analyzed in a short space of time.

Examples of morphometric parameters which may be analyzed according to this aspect of the present invention include, but are not limited to length, persistence, velocity, lamellar activity and directionality. This may be effected using an appropriate microscope operating program and image acquisition software as illustrated in the Examples section hereinbelow.

The method of the present invention may be exploited to screen for agents capable of affecting cell migration.

Thus, according to another aspect of the present invention, there is provided a method of screening for a cell migration affecting agent, the method comprising:

(a) treating a cell with a pharmaceutical agent;

(b) contacting the cell with a plurality of polystyrene non-fluorescent beads so as to generate a migratory track; and

(c) analyzing at least one morphometric parameter of the migratory track wherein a change of the at least one morphometric parameter of the migratory track as compared to a migratory track from a non-treated cell is indicative of a migration affecting agent.

Exemplary pharmaceutical agents which may be screened according to this aspect of the present invention include but are not limited to polynucleotides, polypeptides, carbohydrates, chemicals and a combination of same. The agent may be a known drug or an agent which function is unknown (or at least unknown in the context of cell migration). Accordingly, cells may be “treated” with pharmaceutical agents by incubation, transfection with an expression plasmid encoding a gene of interest, or infection with a virus encoding a genes of interest. In order to screen for agents which up-regulate cell migration, it is preferable to use cells which are non- or low-migratory (e.g. MCF-7 cells). In order to screen for agents which down-regulate cell migration, it is preferable to use cells which are migratory or highly migratory (e.g. MDA-MB-231 cells).

As described in Example 3 hereinbelow, the screening method of the present invention uncovered numerous promigratory polypeptides including Ribosomal protein L14E family (RPL14E) [GI: 20810535]; Eukaryotic elongation factor gamma (EEF1gamma) [GI: 40226406]; Zinc finger protein 36 (C3Htype-like 1, ZFL36L1) [GI: 15812179]; Nucleotide diphosphate kinase (DR-nm23) [GI: 12652978]; Homo Sapiens chromosome 1 clone RP4-7 [GI: 22038620]; BIRC5 [GI: 21707886]; Milk fat globule EGF factor 8 protein (MFGE8) [GI: 13177647]; PKC zeta [GI: 14165514]; Rho GDI alpha (ARHGDIA) [GI: 20149550]; ErbB3 [GI: 12803738]; HOX B7 [GI: 15929846]; Chemokine CXL-ligand 6 (SCYB6) [GI: 15489286]; and Calcium binding protein (CHP) [GI: 6005730].

Modulation of the promigratory polypetptides of the present invention may be used to treat disorders associated with cell migration.

Thus, the present invention provides methods of treating medical conditions associated with cell migration.

As used herein, the term “treating” refers to preventing, alleviating or diminishing a symptom associated with a cell migration-related disease. Preferably, treating cures, e.g., substantially eliminates, the symptoms associated with the migration-related disease.

As used herein the term “subject” refers to any (e.g., mammalian) subject, preferably a human subject.

Medical conditions which would benefit from an upregulation of the promigratory polypeptides of the present invention include any conditions associated with tissue damage such as wound healing, a spinal cord injury, brain injury, brain trauma, a neuronal disease or disorder and inflammatory disorders.

Medical conditions which would benefit from a downregulation of promigratory polypeptides include inflammatory disorders, cancer (e.g. cancer invasiveness) and cancer metastasis (e.g. breast cancer metastasis).

As used herein the phrase “inflammatory disorders” includes but is not limited to chronic inflammatory diseases and acute inflammatory diseases. Examples of such diseases and conditions are summarized infra.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med. Sci. 2000 April; 319 (4):234), myasthenic diseases, Lambert-Eaton myasthenic syndrome (Takamori M. Am J Med. Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann NY Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns. M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann NY Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3): 178), antiphospholipid syndrome (Flamholz R. et al., J Clin Apheresis 1999; 14 (4):171), antibody-induced heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns MP. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg AJ. J Clin Neurosci. 2000 May; 7 (3):191); Guillain-Barre syndrome and autoimmune neuropathies (Kusunoki S. Am J Med. Sci. 2000 April; 319 (4):234), myasthenia, Lambert-Eaton myasthenic syndrome (Takamori M. Am J Med. Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann NY Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly CJ. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann NY Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases (Including Cancer Metastases)

Examples of cancer include but are not limited to carcinoma, lymphoma, blastoma, sarcoma, and leukemia. Particular examples of cancerous diseases include but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

As mentioned herein above, the method of the present invention is effected by administering an agent capable of modulating the activity and/or expression of the migratory polypeptides of the present invention.

As used herein, the term modulating refers to up-regulating or down-regulating. It will be appreciated that the promigratory polypeptides of the present invention may be part of a pathway and thus it may be possible to modulate the polypeptides at a stage further upstream or downstream in the pathway of the promigratory polypeptide. Thus, for example an upstream activator of ErbB3 may be its ligand such as neuregulin. Similarly, a downstream effector of ErbB3 may be PI3K

Methods of treating conditions associated with cell migration may be effected by administering polynucleotides which encode the promigratory polypeptides of the present invention or active portion thereof (as explained below). These may be administered in vivo or ex vivo as further described herein below.

According to one aspect the promigratory polypeptide is RPL14. Agents capable of up-regulating the expression of RPL14 include polynucleotides comprising a nucleic acid sequence encoding an RPL14 polypeptide.

As used herein, the phrase “RPL14 polypeptide” refers to at least an active portion of RPL14. Preferably the RPL14 polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI:20810535.

According to another aspect the promigratory polypeptide is ZFL36L1. Agents capable of up-regulating the expression of ZFL36L1 include polynucleotides comprising a nucleic acid sequence encoding a ZFL36L1 polypeptide.

As used herein, the phrase “ZFL36L1 polypeptide” refers to at least an active portion of ZFL36L1. Preferably the ZFL36L1 polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 15812179.

According to yet another aspect the promigratory polypeptide is EEF1 gamma Agents capable of up-regulating the expression of EEF1 gamma include polynucleotides comprising a nucleic acid sequence encoding a EEF1 gamma polypeptide.

As used herein, the phrase “EEF1 gamma polypeptide” refers to at least an active portion of EEF1 gamma. Preferably the EEF1 gamma polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 40226406.

According to yet another aspect the promigratory polypeptide is Nucleotide diphosphate kinase. Agents capable of up-regulating the expression of Nucleotide diphosphate kinase (DR-nm23) include polynucleotides comprising a nucleic acid sequence encoding a Nucleotide diphosphate kinase polypeptide.

As used herein, the phrase “DR-nm23 polypeptide” refers to at least an active portion of nucleotide diphosphate kinase. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 12652978.

According to yet another aspect the promigratory polypeptide is Homo Sapiens chromosome 1 clone RP4-7. Agents capable of up-regulating the expression of Homo Sapiens chromosome 1 clone RP4-7 include polynucleotides comprising a nucleic acid sequence encoding a Homo Sapiens chromosome 1 clone RP4-7 polypeptide.

As used herein, the phrase “Homo Sapiens chromosome 1 clone RP4-7 poloypeptide” refers to at least an active portion of nucleotide diphosphate kinase. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 22038620.

According to yet another aspect the promigratory polypeptide is BIRC5. Agents capable of up-regulating the expression of BIRC5 include polynucleotides comprising a nucleic acid sequence encoding a BIRC5 polypeptide.

As used herein, the phrase “BIRC5 polypeptide” refers to at least an active portion of BIRC5 (i.e., a portion having promigratory activity). Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 21707886.

According to yet another aspect the promigratory polypeptide is Milk fat globule EGF factor 8 protein. Agents capable of up-regulating the expression of Milk fat globule EGF factor 8 protein (MFGE8) include polynucleotides comprising a nucleic acid sequence encoding a MFGE8 polypeptide.

As used herein, the phrase “MFGE8 polypeptide” refers to at least an active portion of MFGE8. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 13177647.

According to yet another aspect the promigratory polypeptide is PKC zeta. Agents capable of up-regulating the expression of PKC zeta include polynucleotides comprising a nucleic acid sequence encoding a PKC zeta polypeptide.

As used herein, the phrase “PKC zeta polypeptide” refers to at least an active portion of PKC zeta. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 14165514.

According to yet another aspect the promigratory polypeptide is Rho GDI alpha. Agents capable of up-regulating the expression of Rho GDI alpha (ARHGDIA) include polynucleotides comprising a nucleic acid sequence encoding a Rho GDI alpha polypeptide.

As used herein, the phrase “Rho GDI alpha polypeptide” refers to at least an active portion of Rho GDI alpha (i.e., a portion having promigratory activity). Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 20149550.

According to yet another aspect the promigratory polypeptide is ErbB3. Agents capable of up-regulating the expression of ErbB3 include polynucleotides comprising a nucleic acid sequence encoding a ErbB3 polypeptide.

As used herein, the phrase “ErbB3 polypeptide” refers to at least an active portion of ErbB3. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 12803738.

According to yet another aspect the promigratory polypeptide is HOX B7. Agents capable of up-regulating the expression of HOX B7 include polynucleotides comprising a nucleic acid sequence encoding a HOX B7 polypeptide.

As used herein, the phrase “HOX B7 polypeptide” refers to at least an active portion of HOX B7. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 15929846.

According to yet another aspect the promigratory polypeptide is Chemokine CXL-ligand 6. Agents capable of up-regulating the expression of Chemokine CXL-ligand 6 (SCYB6) include polynucleotides comprising a nucleic acid sequence encoding a SCYB6 polypeptide.

As used herein, the phrase “SCYB6 polypeptide” refers to at least an active portion of SCYB6. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 15489286.

According to yet another aspect the promigratory polypeptide is calcium binding protein (CHP). Agents capable of up-regulating the expression of Calcium binding protein (CHP) include polynucleotides comprising a nucleic acid sequence encoding a CHP polypeptide.

As used herein, the phrase “CHP polypeptide” refers to at least an active portion of CHP. Preferably the nucleotide diphosphate kinase polypeptide is at least 50% homologous, more preferably at least 60% homologous, more preferably at least 70% homologous, more preferably at least 80% homologous, and most preferably at least 90% homologous to the polypeptide sequence encoded by the polynucleotide sequence as set forth in GI: 6005730.

The term “nucleic acid sequence” refers to a deoxyribonucleic acid sequence composed of naturally-occurring bases, sugars and covalent internucleoside linkages (e.g., backbone) as well as oligonucleotides having non-naturally-occurring portions which function similarly to respective naturally-occurring portions. Such modifications are enabled by the present invention provided that recombinant expression is still allowed.

Preferably the nucleic acid sequence encodes for the active portion of the promigratory polypeptide. Methods of identifying the nucleic acid sequence which encodes for such active portion include the assay of the present invention and other migration assays known in the art.

A nucleic acid sequence according to this aspect of the present invention can be a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

In order to up-regulate the expression of RPL14, polynucleotides encoding same are ligated into nucleic acid expression vectors, such that the polynucleotide sequence is under the transcriptional control of a cis-regulatory sequence (e.g., promoter sequence).

The expression vector according to this embodiment of the present invention may include additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide expressed from the expression vector of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

A particularly preferred method of administering the pro-migratory polypeptides of the present invention is by gene therapy.

Gene therapy as used herein refers to the transfer of genetic material (e.g. DNA or RNA) of interest into a host to treat or prevent a genetic or acquired disease or condition or phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ. The cells may be autologous or non-autologous to the subject. Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronado, Calif.).

These genetically altered cells have been shown to express the transfected genetic material in situ.

To confer specificity, the nucleic acid constructs used to express the polypeptides of the present invention may comprise cell-specific promoter sequence elements.

Introduction of nucleic acids by infection in both in vivo and ex vivo gene therapy offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in may cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art.

It will be appreciated that when the promigratory polypeptide is a secreted polypeptide e.g. Milk fat globule EGF factor 8 protein (MFGE8), then it is possible to upregulate its expression by administration of the polypeptide itself.

Administration of a polypeptide encompasses native polypeptides (either degradation products, synthetically synthesized polypeptides or recombinant polypeptides) and peptidomimetics (typically, synthetically synthesized polypeptides), as well as as peptoids and semipeptoids which are polypeptide analogs, which may have, for example, modifications rendering the polypeptides more stable while in a body or more capable of penetrating into cells. Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

Polypeptides of present invention can be biochemically synthesized such as by using standard solid phase techniques. These methods include exclusive solid phase synthesis, partial solid phase synthesis methods, fragment condensation, classical solution synthesis. These methods are preferably used when it cannot be produced by recombinant techniques (i.e., not encoded by a nucleic acid sequence) and therefore involves different chemistry or when short peptides are synthesized.

Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Synthetic polypeptides can be purified by preparative high performance liquid chromatography [Creighton T. (1983) Proteins, structures and molecular principles. W H Freeman and Co. N.Y.] and the composition of which can be confirmed via amino acid sequencing.

Recombinant techniques are preferably used to generate the isolated polypeptides of the present invention since these techniques are better suited for generation of relatively long polypeptides (e.g., longer than 20 amino acids) and large amounts thereof. Such recombinant techniques are described hereinabove and further described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

As mentioned hereinabove, modulation of the pro-migratory polypeptides of the present invention may also be down-regulated to treat medical conditions associated with cell migration.

Down-regulating the function or expression of the pro-migratory polypeptides of the present invention can be effected at the RNA level or at the protein level. According to one embodiment of this aspect of the present invention the agent is an oligonucleotide capable of specifically hybridizing (e.g., in cells under physiological conditions) to a polynucleotide encoding the pro-migratory polypeptide.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

A small interfering RNA (siRNA) molecule is another example of an agent capable of downregulating the expression of the promigratory polypeptides of the present invention. RNA interference is a two-step process. During the first step, which is termed the initiation step, input dsRNA is digested into 21-23 nucleotide (nt) small interfering RNAs (siRNA), probably by the action of Dicer, a member of the RNase III family of dsRNA-specific ribonucleases, which cleaves dsRNA (introduced directly or via an expressing vector, cassette or virus) in an ATP-dependent manner. Successive cleavage events degrade the RNA to 19-21 bp duplexes (siRNA), each strand with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al., (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs, which would generate more siRNAs, or by replication of the siRNAs formed. Alternatively or additionally, amplification could be effected by multiple turnover events of the RISC [Hammond et al., Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the promigratory polypeptide (e.g. RPL14) polynucleotide sequence target is scanned downstream for AA dinucleotide sequences. Occurrence of each AA and the 3′ adjacent 19 nucleotides is recorded as potential siRNA target sites.

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites that exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating the expression of the promigratory polypeptides of the present invention is a DNAzyme molecule capable of specifically cleaving its encoding polynucleotide. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 94:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of Chronic Myelogenous Leukemia (CML) and Acute Lymphocytic Leukemia (ALL).

Another agent capable of downregulating the expression of the promigratory polypeptides of the present invention is a ribozyme molecule capable of specifically cleaving its encoding polynucleotide. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of downregulating the function of a promigratory polypeptide of the present invention is via triplex forming oligonucleotides (TFOs). In the last decade, studies have shown that TFOs can be designed which can recognize and bind to polypurine/polypirimidine regions in double-stranded helical DNA in a sequence-specific manner. Thus the DNA sequence encoding the polypeptide of the present invention can be targeted thereby down-regulating the polypeptide.

The recognition rules governing TFOs are outlined by Maher III, L. J., et al., Science (1989) 245:725-730; Moser, H. E., et al., Science (1987) 238:645-630; Beal, P. A., et al., Science (1991) 251:1360-1363; Cooney, M., et al., Science (1988) 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer (2003) J Clin Invest; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch (2002), BMC Biochem, September 12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and subsequent formation of the triple helical structure with the target DNA, induces steric and functional changes, blocking transcription initiation and elongation, allowing the introduction of desired sequence changes in the endogenous DNA and results in the specific downregulation of gene expression. Examples of such suppression of gene expression in cells treated with TFOs include knockout of episomal supFGl and endogenous HPRT genes in mammalian cells (Vasquez et al., Nucl Acids Res. (1999) 27:1176-81, and Puri, et al., J Biol Chem, (2001) 276:28991-98), and the sequence- and target-specific downregulation of expression of the Ets2 transcription factor, important in prostate cancer etiology (Carbone, et al., Nucl Acid Res. (2003) 31:833-43), and the pro-inflammatory ICAM-1 gene (Besch et al., J Biol Chem, (2002) 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res (2000);28:2369-74).

Additionally, TFOs designed according to the abovementioned principles can induce directed mutagenesis capable of effecting DNA repair, thus providing both downregulation and upregulation of expression of endogenous genes [Seidman and Glazer, J Clin Invest (2003) 112:487-94]. Detailed description of the design, synthesis and administration of effective TFOs can be found in U.S. Patent Application Nos. 2003 017068 and 2003 0096980 to Froehler et al., and 2002 0128218 and 2002 0123476 to Emanuele et al., and U.S. Pat. No. 5,721,138 to Lawn.

As mentioned hereinabove, down regulating the function of a promigratory polypeptide of the present invention can also be affected at the protein level.

Thus, another example of an agent capable of downregulating a promigratory polypeptide of the present invention is an antibody or antibody fragment capable of specifically binding to it, preferably to its active site, thereby preventing its function.

As used herein, the term “antibody” refers to a substantially intact antibody molecule.

As used herein, the phrase “antibody fragment” refers to a functional fragment of an antibody that is capable of binding to an antigen.

Suitable antibody fragments for practicing the present invention include, inter alia, a complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, and an F(ab′)2.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker.

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

Methods of generating monoclonal and polyclonal antibodies are well known in the art. Antibodies may be generated via any one of several known methods, which may employ induction of in vivo production of antibody molecules, screening of immunoglobulin libraries (Orlandi, R. et al. (1989). Cloning immunoglobulin variable domains for expression by the polymerase chain reaction. Proc Natl Acad Sci USA 86, 3833-3837; and Winter, G. and Milstein, C. (1991). Man-made antibodies. Nature 349, 293-299), or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler, G. and Milstein, C. (1975). Continuous cultures of fused cells secreting antibody of predefined specificity. Nature 256, 495-497; Kozbor, D. et al. (1985). Specific immunoglobulin production and enhanced tumorigenicity following ascites growth of human hybridomas. J Immunol Methods 81, 31-42; Cote R J. et al. (1983). Generation of human monoclonal antibodies reactive with cellular antigens. Proc Natl Acad Sci USA 80, 2026-2030; and Cole, S. P. et al. (1984). Human monoclonal antibodies. Mol Cell Biol 62, 109-120).

It will be appreciated that for human therapy or diagnostics, humanized antibodies are preferably used. Humanized forms of non-human (e.g., murine) antibodies are genetically engineered chimeric antibodies or antibody fragments having (preferably minimal) portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody (see, for example: Jones, P. T. et al. (1986). Replacing the complementarity-determining regions in a human antibody with those from a mouse. Nature 321, 522-525; Riechmann, L. et al. (1988). Reshaping human antibodies for therapy. Nature 332, 323-327; Presta, L. G. (1992b). Curr Opin Struct Biol 2, 593-596; and Presta, L. G. (1992a). Antibody engineering. Curr Opin Biotechnol 3(4), 394-398).

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed essentially as described (see, for example: Jones et al. (1986); Riechmann et al. (1988); Verhoeyen, M. et al. (1988). Reshaping human antibodies: grafting an antilysozyme activity. Science 239, 1534-1536; and U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries (Hoogenboom, H. R. and Winter, G. (1991). By-passing immunisation. Human antibodies from synthetic repertoires of germline VH gene segments rearranged in vitro. J Mol Biol 227, 381-388; Marks, J. D. et al. (1991). By-passing immunization. Human antibodies from V-gene libraries displayed on phage. J Mol Biol 222, 581-597; Cole et al. (1985), Monoclonal Antibodies and Cancer Therapy, Alan R. Liss, Inc., pp. 77-96; and Boerner, P. et al. (1991). Production of antigen-specific human monoclonal antibodies from in vitro-primed human splenocytes. J Immunol 147, 86-95). Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016; Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93).

The agents of the present invention can be provided to the individual per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide or polynucleotide preparation, which is accountable for the biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

As used herein the term “about” refers to +10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Establishment of a High-Throughput PKT Assay

The PKT assay was amended and recalibrated according to the methods described below so that it could be used to measure the migration of a wide variety of cells with a high throughput, whilst obtaining maximal information on a number of migration parameters.

Materials and Methods

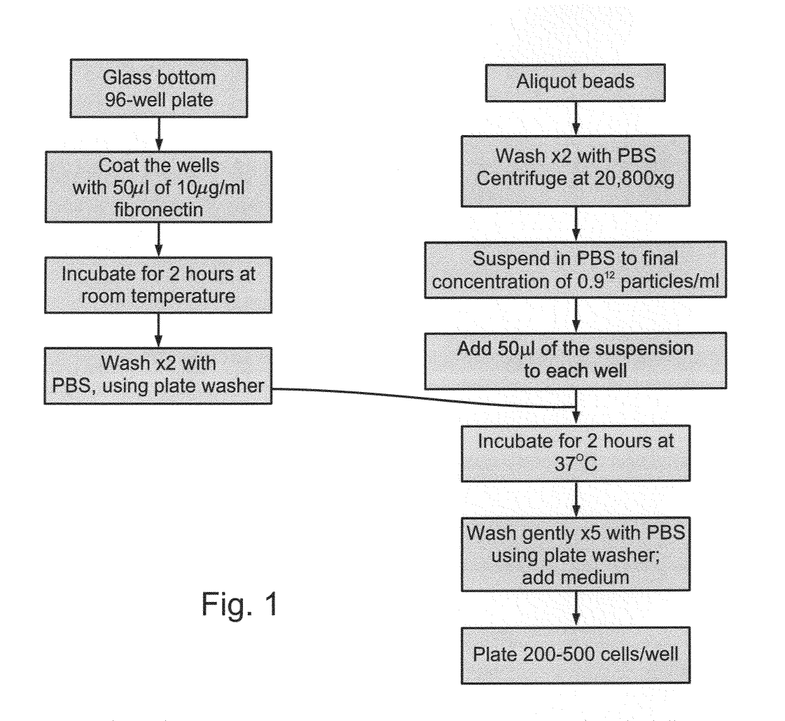

Preparation of 96-wellplates: Glass-bottom 96-well plates (Cat. # 7706-2370, Whatmann, Inc., Clifton, N.J., USA) were treated for 2 hours at room temperature with 50 μl of 10 μg/ml fibronectin solution dissolved in PBS (Fibronectin, F-1141; Sigma Chemical Co., St Louis, Mo., USA). The wells were then washed twice with PBS and coated with 340 nm diameter white polystyrene latex beads (Product no. 2-300; batch no. 1344); Interfacial Dynamics Corporation-Molecular Probes Microspheres Technologies, USA. The bead suspension (3.2 ml) was centrifuged for 5 minutes at 20,800×g, and resuspended in 4 ml PBS by vortex, until all visible bead clumps were dispersed. The sedimentation procedure was then repeated, after which the beads were resuspended in 7 ml PBS, to a final concentration of 0.9¹² particles/ml. Aliquots of 70 μl of the bead suspension were added to each well, pre-coated with fibronectin, in the 96-well plate, and incubated at 37° C. for 2 hours, followed by gentle washing (×5 times) with PBS using a plate washer (Colombus Plus, Tecan, Switzerland). Before cells were plated, the PBS was replaced with 50 μl culture medium suitable for the particular cell type used in the assay (see FIG. 1).

Cell preparation for the phagokinetic track assay: MCF7 (ATCC-HTB-22), MDA-MB-231 (ATCC-HBT-26), and B16-F10 (ATCC-CRL-6475) cells were grown in Dulbeco's Modified Eagle's Medium (DMEM). H1299 cells (ATCC-CRL-5803) were grown in RPMI-1640. Both culture media were supplemented with 10% FCS, 2 mM glutamine, 100 international units/ml penicillin and 100 μg/ml streptomycin (Biological Industries, Beit Haemek, Israel), and placed in a 5% CO₂ humidified incubator kept at 37° C. For the PKT assay, 200-400 cells (a 50 μl volume) were cultured in each well. Depending on the track dimensions, the number of the plated cells was calibrated to maximize the number of single cell tracks, yet minimize the number of intersecting tracks. For MCF7 cells, ˜400 cells/well was found to be optimal; for B16-F10 cells, 300 cells/well; and for the more motile MDA-MB-231 and H1299 cells, ˜200 cells/well were plated.

Phagokinetic track assay: In order to characterize the motile behavior of the various cell lines, MCF7, MDA-MB-231, B16-F10 and H1299 cells were plated on a monolayer of polystyrene beads. All were incubated at 37° C., 5% CO₂ for 7 hours, except for the H1299 cells, which were incubated for 5 hours. The first three cell lines were grown in DMEM, and H1299 cells were grown in RPMI 1640 medium, Both media were supplemented with 10% FCS and penicillin/streptomycin antibiotic. At the end of the incubation period, cells were fixed with 3% PFA. Images were taken with a 10× objective, using the autofocus screening microscope system.

Automated microscopy: Commercially-available plates display large deviations from a plane, often exceeding 200 μm, which require adjustment of the focal plane for every image. Commonly-used autofocusing procedures, which are based on finding the highest contrast image among a focal series, are slow and not robust, particularly when low-contrast images, such as those yielded by a monolayer of micro-beads, are used. The phagokinetic tracks were therefore recorded using a cell-screening microscope described in detail (Paran et al., Methods in Enzymology, in press). The rapid, automated collection of sharp, in-focus PKT images was aided by a laser autofocus device (Liron et al., 2006, J Microsc 221, 145-51). The laser autofocus device enables precise location of the substrate surface, and guarantees sharp definition of track boundaries.

The microscope operating program and the image acquisition software was written (In-house) as an application within the UCSF Priism environment (http://msg.ucsf.edu/ive). The microscope includes a computer-controlled plate scanning stage (Prior, Cambridge UK) that defines the pattern of coverage of the well area by the individual images. For this application, images were taken using a 10×/0.4 objective, and adjacent fields were stitched together, forming a montage of the entire well, or any desired fraction of the well area.

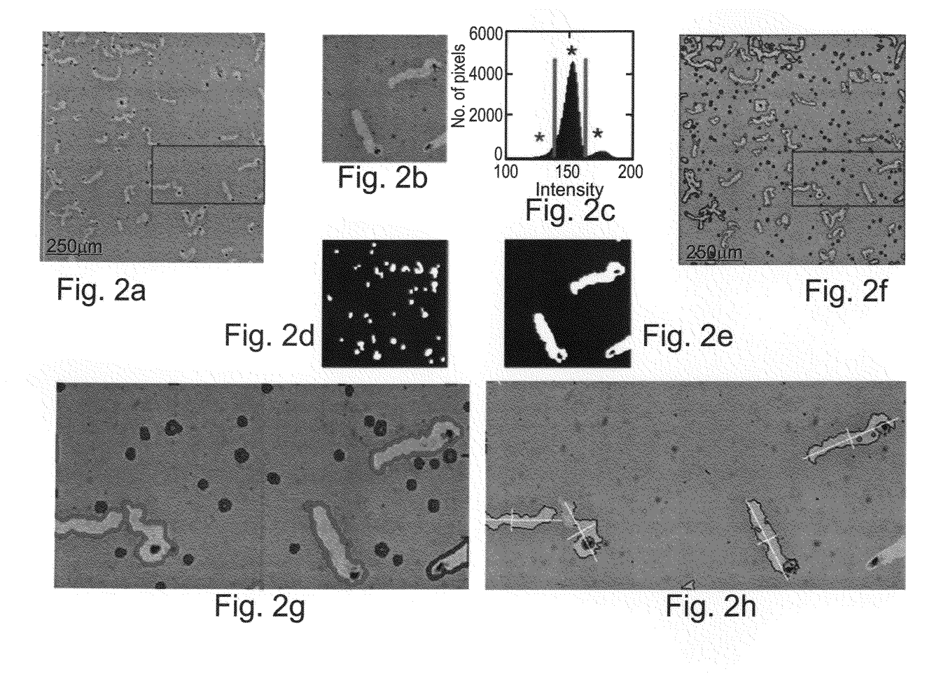

Image processing: Non-homogenous illumination and shadows, commonly caused by the narrow well walls, were minimized by a light diffuser inserted above the plate; images were further corrected by post-acquisition processing. Averaging of background intensity was calculated and subtracted to flatten the image (high-pass filtration), which created images with high contrast for the tracks. Montage images provided not only an easy way to visualize impressions of cell migration within the well, but also merged track fragments from adjacent images, in order to minimize the number of incomplete “border tracks” rejected by the assay (see below). In order to locate the tracks, image smoothing was used in order to average high-frequency noise, and facilitate binary segmentation (FIGS. 2A-H). This was followed by adaptive threshold and connected component analyses.

In the histogram of the intensity levels of pixels in the images analyzed, three distinct intensity ranges were seen: a high peak, composed of background gray pixels (FIG. 2C, black asterisk); a low peak, indicating high pixel intensity, contributed by bead-free bright tracks (FIG. 2C, blue asterisk); and the cell pixels, including some debris, indicated by low-intensity pixels that do not create a peak (FIG. 2C, red asterisk). By applying two binary thresholds the dark cells (pixels below the low threshold; FIG. 2D), and the bright tracks (pixels above the high threshold; FIG. 2E) were identified. By means of connected component analysis for the thresholded pixels, the tracks as individual objects could be defined (FIG. 2E). Objects that were either too small (e.g., cell debris), too large (e.g., a scratch in the bead monolayer, or a cluster of intersecting tracks), or located on the border of the image, were discarded (FIG. 2F, segments outlined in blue). The “legal” objects (outlined in red) defined the regions of interest; however they displayed blurred borders (FIG. 2G). Therefore, fine details of track shape were calculated using multiscale segmentation analysis (FIG. 2H). A slightly-modified multi-scale segmentation algorithm (Sharon et al., 2001, IEEE, 1469-1476) was applied to each region of interest. A pyramid structure over the image was constructed by means of a recursive process of weighted aggregation, based on pixel similarity. Coarse scale measurements were calculated on this structure. These measurements yielded a hierarchical averaging of intensities, and texture and shape descriptors, which were used to classify the segments identifying cells (by means of the dark, phagocytosed beads in the perinuclear region), tracks, and the beaded carpet background. Since a single track may emerge in more than one multiscale segment, we follow by a step of joining all touching cells and tracks segments. Track morphological parameters were then determined. Tracks without cells, or those containing two or more cells, were discarded. Since multiple-cell tracks may result from tracks merging, or cell divisions at unknown times, normalization of the track area according to the number of enclosed cells is not justified. The morphometric parameters calculated for each PKT are defined and described in Table 1 hereinbelow.

TABLE 1

| Parameter | | |
|---|---|---|
| annotation | | |
| [dimensions] | Explanation | Measured/calculated by |
| A [μm²] | PKT area [cell area subtracted] | Matlab: regionprops.area |
| | | Average cell area value was subtracted |
| | | manually from the PKT area value. |
| P [μm] | Track perimeter | Matlab: regionprops.perimeter |
| R | Roughness, R = P²/(4p*A) | Calculated from A and P |
| D_{L}, D_{S} [μm] | Major (long) and minor (short) axes | Matlab: |
| | of best fit ellipsoid, calculated from | regionprops.MajorAxisLength/ |
| | second moments about center of | matlab: regionprops.MinorAxisLegth |
| | area | |
| X | Axial Ratio, X = D_{L}/D_{S} | Calculated from D_{L}, D_{S} |
| A_{CH} [μm²] | Convex hull area is the region | Matlab: regionprops.ConvexArea |
| | between the smallest convex set that | |
| | contains all the outside points of the | |
| | track | |
| S | Solidity = A/A_{CH} | Calculated from A, ACH |
| L [μm] | Main track skeleton length | Manually defined; cell length values |
| | | are subtracted |
| B [μm] | Sum of skeleton branches | Manually defined |
| E [μm] | End- to-end distance of track | Calculated by the program |
| | skeleton | |
| T [h] | Total migration time (hours) | Measured |
| V_{E} | Effective velocity = E/T | Calculated from E and T |
| V_{M} | Migration velocity = (L + B)/T | Calculated from L, B and T |

Statistical analysis: Since the distribution of track parameters displays wide variability and is therefore not considered to be normal, the results were reported as both mean±standard deviation, as well as median, with half of the “interquartile range” [(75^{th} percentile-25^{th} percentile)/2]. Differences between control and treated cultures were evaluated with the Two-sample Kolmogorov-Smirnov goodness-of-fit hypothesis test. A p value of <0.05 was considered to be statistically significant.

Pearson's correlation test was conducted. It ranges from +1 to −1. A correlation of +1 means that there is a perfect positive linear relationship between two tested variables and a correlation of −1 means perfect negative linear relationship. A p value of <0.0014 was considered to be statistically significant.

Results

Selection of beads for the high-throughput PKT assay: A variety of micro-beads, displaying a wide range of dimensions and chemical properties, were tested. The differences in bead dimension and surface chemistry greatly affect their capacity to form a uniform monolayer, to firmly attach to the surface of the well and yet to be effectively cleared by migrating cells. Moreover, the optimal bead for use in PKT assays was also dependent on the cell type as shown in Table 2 hereinbelow.

TABLE 2

| | Bead | Surface group and | | | Bead | | | |
|---|---|---|---|---|---|---|---|---|
| Product | diameter | charge content μEq/g | | | attachment | Monolayer | PKT | |
| #/Batch # | (nm) | Carboxyl | Sulfate | Aldehyde | strength | quality | formation | Contrast |
| 2-300/1867 | 310 | 23.9 | N/A | — | +++ | Homogeneous | — | N/R |
| 2-300/1041 | 320 | 201.2 | N/A | — | ++ | Homogeneous | **Clear | ++ |
| 12-300/1178 | 330 | — | N/A | 11.5 | +++ | Homogeneous | — | N/R |
| 2-300/2431 | 330 | 162.1 | 9.4 | — | ++ | Homogeneous | *Clear | +++ |
| 1-300/1053 | 340 | — | 3.6 | — | Beads produce aggregates following centrifugation and | | | |
| | | | | | were hard to homogenize | | | |
| 2-300/1344 | 340 | 184.7 | N/A | — | ++ | Homogeneous | *Clear | +++ |
| 1-300/401 | 350 | — | 0.6 | — | Beads produce aggregates following centrifugation and | | | |
| | | | | | were hard to homogenize | | | |
| 1-300/1955 | 350 | — | 2.1 | — | Beads produce aggregates following centrifugation and | | | |
| | | | | | were hard to homogenize | | | |
| 1-400/1915 | 350 | — | 6.0 | — | Beads produce aggregates following centrifugation and | | | |
| | | | | | were hard to homogenize | | | |
| 2-400/1049 | 400 | 91.4 | N/A | — | +++ | Homogeneous | **Clear | +++ |

| | | | | | | | |
|---|---|---|---|---|---|---|---|
| 2-1000/1685 | 1000 | 446.0 | N/A | — | ± | Low beads | Beads were floating |
| | | | | | | density | close to the surface and |
| | | | | | | | tracks were not |
| | | | | | | | produced by the cells |

All the beads are negatively charged
N/A = Not Applicable
N/R = Not Relevant-PKT s were not formed
*Suitable for very wide range of cell types.
**Suitable for specific cell lines only (H1299, Ref-52).
+ Low
++ Medium
+++ High

The beads that were found to be suitable for PKT assays applied to most cell types were 340 nm diameter, surfactant-free Carboxylated Modified Latex (CML) white polystyrene beads, negatively charged due to carboxylate groups on their surface, with a charge content of 184.7 μEq/g. These beads form a homogenous and visible monolayer; moreover, their attachment to the substrate is firm enough to prevent spontaneous detachment, but still weak enough to be removed by migrating cells.

The surface chemistry of the beads had a strong effect on the PKT assay: beads with an aldehyde-modified surface attached firmly to the substrate, and could not be removed by migrating cells. Beads with a sulfated surface tended to form aggregates following centrifugation and suspension, thus yielding a non-uniform monolayer. Carboxylated beads, with or without sulfate groups, tended to form rather homogenous suspensions after centrifugation.

Moreover, phagokinetic tracks made by smaller beads (<300 nm in diameter) have low contrast and were hard to visualize. Large beads (˜1000 nm in diameter) tended to detach from the surface and then spontaneously reattach, resulting in poorly-defined tracks. Thus, it appeared that beads with diameters of 300-400 nm were an optimal size for the automated PKT assay. The 400 nm beads produced high-contrast phagokinetic tracks that could be readily visualized with the naked eye, while smaller beads (˜300-350 nm) produced lower-contrast tracks that could be readily visualized and segmented, following computerized contrast enhancement.

The surface density of the carboxyl groups also affected track formation: a low charge content on the bead's surface (23.9 μEq/g) led to a strong interaction between the bead and the surface, such that many cell types failed to effectively remove the beads as they migrated. Beads with carboxyl groups of intermediate density (91.4 μEq/g) were found to be suitable for assays involving certain cell types, mainly strongly adhering cells (e.g. H1299-non-small cell lung carcinoma- and Ref52-Rat Embryo Fibroblasts) but not others, loosely adhering cells (e.g. MCF7— Breast carcinoma, B16-F10— melanoma). Beads with carboxyl groups with a density of 160-185 μEq/g were found to be optimal for assays applied to a wide range of cell types.

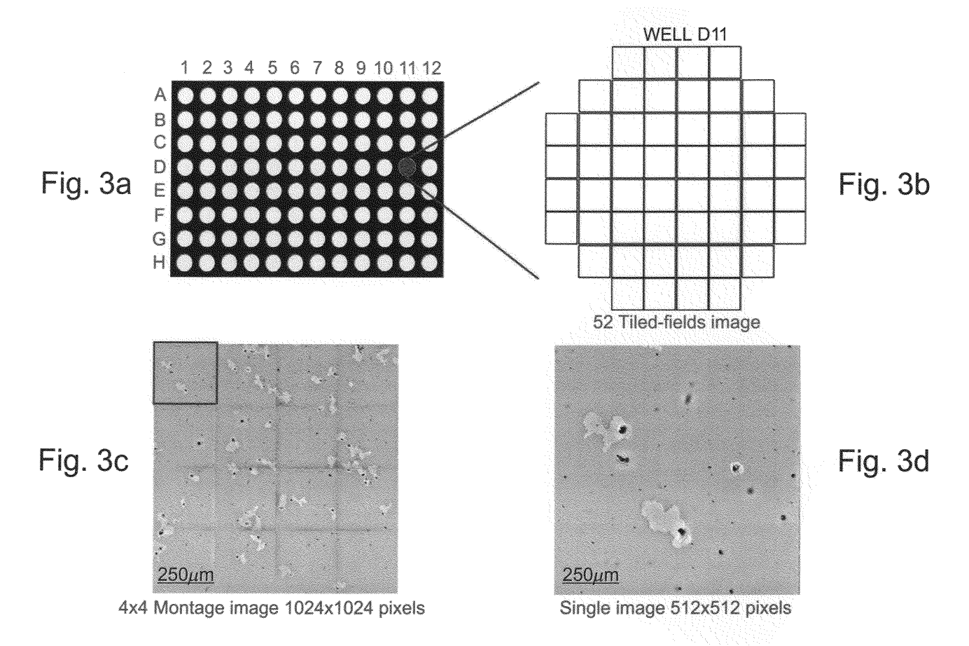

Recording of PKT images using automated microscopy: The computer program, which was developed for the PKT assay of the present invention, controlled the autofocusing and image acquisition steps for all selected fields within each well and for all selected wells in the plate, and stored the resulting image data in a file. Together with each image, all the experimental parameters (e.g. well number, position within the well, exposure time, objective, illumination setting, etc.) were also stored. In order to record the maximum number of complete cell tracks, images of adjacent fields were fused, forming a “seamless” montage in which tracks spanning more than one image were merged. In the experiments reported herein, only images taken from the central region of the 96-well plate (30% of the total well area) were recorded. Typically, 16 images (a 4×4 panel, 1024×1024 pixels in dimension) were prepared and subjected to analysis (FIGS. 3A-D).

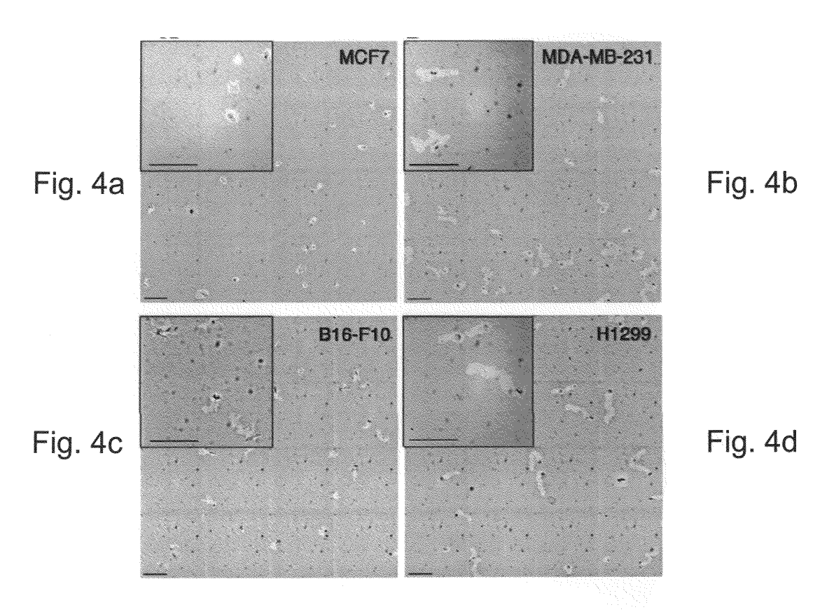

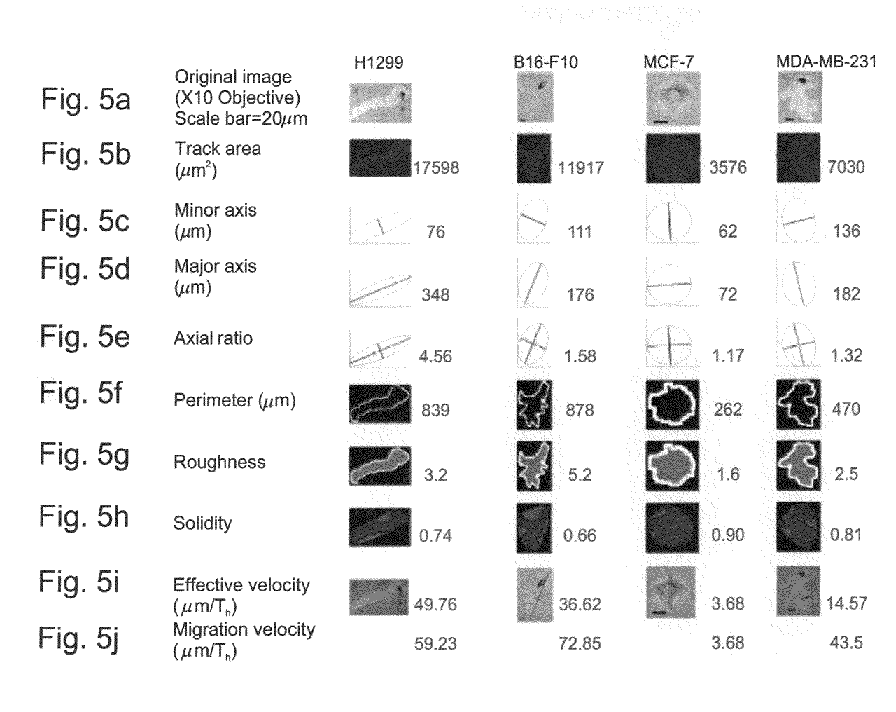

Different cell types produce phagokinetic tracks with distinct characteristics: Using the PKT assay of the present invention, a wide range of cell lines were tested, including: melanomas (B16-F10 and B16-F1), breast carcinomas (MDA-MB-231 and MCF7), rat fibroblasts (REF52, SV80 and NIH3T3), H1299 lung carcinoma, and several types of prostate carcinoma (DU145, PC3 and CL1). Image analyses of the phagokinetic tracks that were generated enabled the present inventors to distinguish between the migratory properties of the various cell lines. Four cell-lines were selected to demonstrate these differences, the MDA-MB231, MCF7, H1299 and B16-F10, displaying distinct migration characteristics (FIGS. 4A-D and Table 3 hereinbelow).

TABLE 3

| | H1299 | B16-F10 | MCF7 | MDA-MB-231 |
|---|---|---|---|---|
| | N = 104 | N = 124 | N = 93 | N = 149 |
| Track area (μm²) | 14000 ± 7600 | 7400 ± 2800 | 4900 ± 2400 | 13500 ± 6300 |
| | [13700//5500] | [7500//2200] | [4400//1700] | [12600//4600] |
| Major axis(μm) | 220 ± 97 | 150 ± 41 | 113 ± 29 | 188 ± 61 |
| | [206//70] | [146//23] | [108//21] | [180//44] |
| Minor axis (μm) | 106 ± 36 | 90 ± 19 | 75 ± 20 | 111 ± 30 |
| | [98//23] | [88//12] | [71//13] | [109//21] |
| Axis ratio | 2.1 ± 0.9 | 1.8 ± 0.5 | 1.5 ± 0.4 | 1.7 ± 0.5 |
| | [1.9//0.5] | [1.7//0.3] | [1.4//0.2] | [1.6//0.3] |
| Perimeter (μm) | 710 ± 330 | 560 ± 161 | 400 ± 150 | 650 ± 260 |
| | [655//220] | [565//130] | [350//100] | [605//180] |
| Solidity | 0.8 ± 0.1 | 0.7 ± 0.08 | 0.8 ± 0.08 | 0.8 ± 0.08 |
| | [0.8//0.08] | [0.7//0.06] | [0.8//0.06] | [0.8//0.06] |
| Roughness | 2.6 ± 1.0 | 3.3 ± 1.0 | 2.4 ± 0.8 | 2.4 ± 0.8 |
| | 2.8//0.9] | [3.5//0.8] | [2.5//0.6] | [2.4//0.6] |
| Effective velocity | 32 ± 13 | 17 ± 6 | 6 ± 3 | 26 ± 10 |
| (μm/h) | [30//0.9] | [17//4] | [6//2] | [25//7] |
| Migration | 44 ± 17 | 40 ± 18 | 6 ± 3 | 58 ± 29 |
| velocity (μm/h) | [43//11] | [38//11] | [6//2] | [54//19] |

[a//b] denotes the median, a, and the value of half of the “interquartile range” (half the difference between the 25^{th} and 75^{th} percentiles), b.

MCF7 cells, for example, migrate poorly, producing only a small, bead-free track surrounding each cell, with an average area of 4,900±2,400 μm² (n=93). The B16-F10 melanoma cells produce branched tracks due to the extension of multiple filopodia; such cells produce narrow, bead-free protrusions along the main migratory path, with an average area of 7,400±2800 μm² (n=124). The MDA-MB-231 cells are metastatic cells with high levels of migratory activity, producing long, wide tracks with an average area of 13,500±6,300 μm² (n=149). H1299 cells are migratory and highly persistent forming tracks with an average area of 14,000±7,600 μm² (n=104).

Additional morphometric parameters were also calculated for each of the cell lines in order to quantify migratory features such as persistence, effective velocity, average migration velocity, lamellar activity and overall directionality. Analysis of these parameters was carried out on tracks produced by each of the cell types (FIGS. 5A-J). Thus, the axial ratio of the track produced by H1299 cells, for example, indicated highly persistent migration (about fourfold higher than that of MCF7 cell, and threefold higher than those of B 16-F 10 and MDA-MB-231 cells). It is noteworthy that migration rate and persistence are clearly distinct features, as manifested by the conspicuous differences between effective velocity and migration velocity. For example, B16-F10 cells exhibit higher migration velocity (72.85 μm/hr) than H1299 cells (59.23 μm/hr), while the latter cell type displays the highest effective velocity (49.76 μm/hr, compared to 36.62 μm/hr in B16-F10 cell), indicating a more persistent migration.

To assess “lateral” lamellar activity, which affects the width and roughness of track borders, the track perimeter was measured and roughness and solidity parameters were calculated. This analysis showed that while the perimeters of track formed by H1299 and B16-F10 cells were nearly the same, the roughness parameter was higher in B16-F10 (5.2) than in H1299 (3.2), indicating a higher level of lamellar activity of the B16-F10 cell. The border roughness also provides evidence on lamellipodial activity. This finding was directly confirmed by time-lapse movies (data not shown.) The solidity parameter, too, provided information about the smoothness of the track border due to lamellar activity: a solidity value of 1, for example, indicated a smooth and persistent track, as was the case in MCF7 cell. Lower solidity values indicated a convoluted and rough track, as was the case in B16-F10 cell.

### Example 2

### The Effects of Cytoskeletal Drugs on Cell Migration as Assayed by the PKT System of the Present Invention

In order to explore the capability of the automated screening system of the present invention to detect changes in specific migratory features induced by chemical perturbations, H1299 cells were treated with various compounds known to affect cell motility. The effect of each drug on the different morphometric parameters was then measured.

Materials and Methods

Cell culture: Cells were plated and incubated for one hour, after which they were treated with 4 μM of either Latrunculin A, 2.5 μM Nocodazole, or 100 ng/ml PMA (Phorbol 12-mirysyate 13-acetate). The cells were kept in the incubator for a total of 5 hours; they were then fixed with 3% paraformaldehyde and washed twice with PBS. Plates were either examined immediately by the screening autofocus microscope, or stored at 4° C. for later inspection.

PKT assay: The PKT assay was performed as described for Example 1.

Results

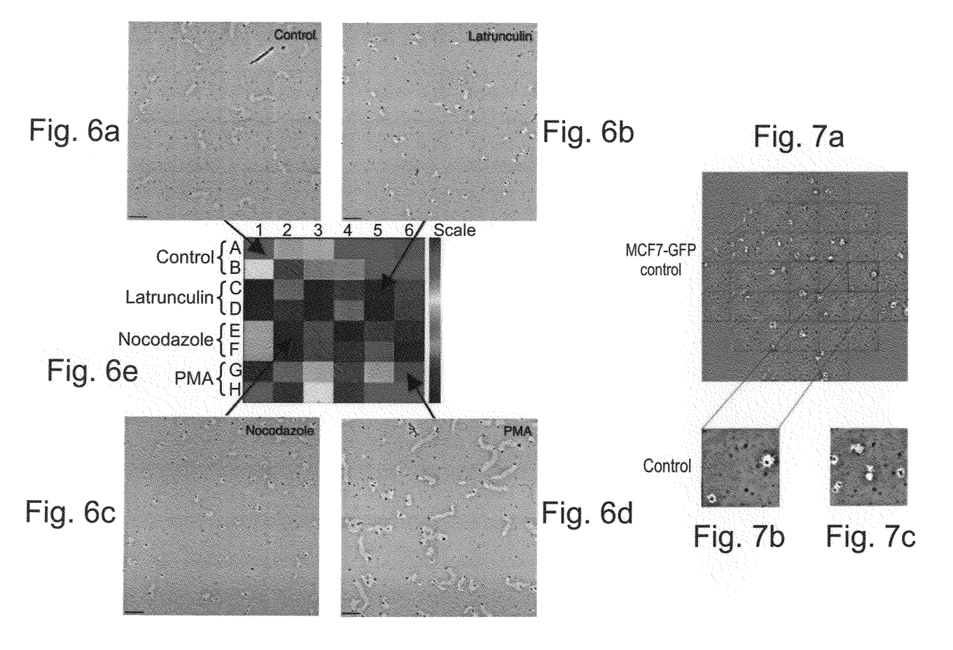

The results are illustrated in FIGS. 6A-E. Each rectangle in the color-coded plot represents one well in the 96-well plate. The color represents the average value of the selected PKT parameter in the particular well. Since the distributions of the values for each PKT parameter did not appear to be normal, the median and percentile values were also calculated for each morphometric parameter (Table 4, hereinbelow).

TABLE 4

| | Control | Latrunculin A | Nocodazole | PMA |
|---|---|---|---|---|
| | n = 421 tracks | n = 602 tracks | n = 588 tracks | n = 380 tracks |
| Track area (μm²) | 12500 ± 7700 | 5900 ± 3500 | 6100 ± 3600 | 14300 ± 9100 |
| | [11300//5600] | [4700//1900] | [5100//2000] | [12800//6400] |
| Major axis (μm) | 200 ± 98 | 121 ± 44 | 123 ± 43 | 220 ± 104 |
| | [180//72] | [103//25] | [111//26] | [200//80] |
| Minor axis (μm) | 100 ± 33 | 79 ± 19 | 78 ± 18 | 108 ± 42 |
| | [90//22] | [73//11] | [75//12] | [96//24] |
| Axis ratio | 2.1 ± 0.9 | 1.5 ± 0.4 | 1.6 ± 0.4 | 2.1 ± 0.9 |
| | [1.8//0.5] | [1.4//0.2] | [1.5//0.3] | [1.8//0.6] |
| Perimeter (μm) | 615 ± 290 | 380 ± 150 | 385 ± 140 | 695 ± 350 |
| | [560//215] | [320//76] | [350//86] | [642//240] |
| Solidity | 0.8 ± 0.1 | 0.9 ± 0.07 | 0.9 ± 0.07 | 0.8 ± 0.1 |
| | [0.8//0.08] | [0.9//0.04] | [0.9//0.04] | [0.8//0.08] |
| Roughness | 3.0 ± 1.1 | 2.0 ± 0.7 | 2.1 ± 0.9 | 2.9 ± 1.4 |
| | [2.3//0.9] | [1.7//0.4] | [1.8//0.4] | [2.6//0.9] |
| Effective velocity | 36 ± 14 | 8 ± 5 | 14 ± 8 | 46 ± 21 |
| (μm/h) | [41//12] | [6//3] | [12//5] | [43//16] |
| Migration velocity | 42 ± 17 | 10 ± 6 | 17 ± 11 | 59 ± 26 |
| (μm/h) | [43//12] | [8//4] | [13//6] | [55//18] |

Comparison of the mean (±standard deviation) and median or percentile values, generally indicated good agreement among the different statistical approaches.

Analysis of the PKT area of H1299 cells treated with the various inhibitors pointed to the usefulness of the quantitative approach. PKT produced by Latrunculin- or Nocodazole-treated cells exhibited reduced areas, axis ratios and migration velocities, while their roughness decreased (p=˜0) compared to the control cells. In the PMA-treated cells, the PKT areas, solidity and roughness parameters, and migration velocities increased (p<0.05), but the major and minor axes, axial ratios, and effective velocities did not significantly differ from those of control cells, suggesting an increase in cell protrusive activity, with frequent changes in direction while migrating.

### Example 3

Retroviral based migration screens in combination with the PKT assay of the present invention were used to analyze the low migratory breast cell line MCF7 over-expressing genes from a BC1000 cDNA library and genes from a MDA-MB-231 cDNA library, known to be involved in breast cancer, to identify migration inducing genes.

Materials and Methods

BC100 cDNA migration screen: 55 genes out of total 1,000 genes that compose the BC1000 library were screened, this library is a collection of full length cDNAs associated with breast cancer development and breast carcinogenesis. The BC1000 gene list is comprised of genes suggested by scientists at the Harvard Institute of Proteomics and from experts in breast cancer research. The cDNAs were cloned into a puromycin selectable retroviral vector-JP1520. The Fifty-five genes (Table 5 herein below) were randomly selected from this library and were generously provided by Joan Brugge's laboratory (Department of Cell Biology, Harvard Medical School, USA).

TABLE 5

| # | GI number | Gene symbol |
|---|---|---|
| 1 | 12803738 | ERBB3 |
| 2 | 15079546 | G1P3 |
| 3 | 14602762 | MDM2 |
| 4 | 15679935 | ENG |
| 5 | 13177647 | MFGE8 |
| 6 | 14250476 | IL1B |
| 7 | 14250621 | EMK1 |
| 8 | 16307254 | SCYA2 |
| 9 | 12653770 | CLCDN4 |
| 10 | 12803364 | BC-2 |
| 11 | 12653114 | GRN |
| 12 | 17390233 | STRIN |
| 13 | 15126675 | CFL1 |
| 14 | 13177717 | DDIT3 |
| 15 | 14165514 | PRKCZ |
| 16 | 12804382 | BFAR |
| 17 | 15929846 | HOXB7 |
| 18 | 13279010 | RAC1 |
| 19 | 15278147 | TRIP10 |
| 20 | 21707886 | BIRCS |
| 21 | 184833 | IGF-1 |
| 22 | 18088238 | BMP4 |
| 23 | 13325245 | BIRC5 |
| 24 | 13905041 | TPD52L2 |
| 25 | 1628549 | EGFR |
| 26 | 4504158 | GRP |
| 27 | 15079240 | IGFBP5 |
| 28 | 4507170 | SPARC |
| 29 | 15930064 | ABS |
| 30 | 12653134 | FADD |
| 31 | 15341773 | LIMK2 |
| 32 | 15489286 | SCYB6 |
| 33 | 14165485 | SPHK1 |
| 34 | 13623610 | STK15 |
| 35 | 10947110 | ARG2 |
| 36 | 20149550 | ARHGDIA |
| 37 | 4757771 | ARHI |
| 38 | 10938017 | CCNB2 |
| 39 | 4757945 | CD83 |
| 40 | 6005730 | CHP |
| 41 | 4758077 | CSK |
| 42 | 4504612 | CYR61 |
| 43 | 11038657 | EDG4 |
| 44 | 15147344 | FGF7 |
| 45 | 5031702 | G3BP |
| 46 | 10834983 | IL6 |
| 47 | 13259537 | KAI1 |
| 48 | 21359833 | PPAP2B |
| 49 | 4505590 | PRDX1 |
| 50 | 4506274 | PTK9 |
| 51 | 18656935 | PTN |
| 52 | 4507266 | STC2 |
| 53 | 4827037 | TPD52 |
| 54 | 4885654 | WNT1 |
| 55 | 20149551 | BCS1L |

[a//b] denotes the median, a, and the value of half of the “interquartile range” (half the difference between the 25^{th} and 75^{th} percentiles), b.

Genes were introduced by retroviral infection to MCF7 cells. Each clone was tested for its impact on cell migration using the PKT assay. As control JP1520-GFP expressing MCF7 cells were also generated. The PKT assay was performed using the 96 wells plates. Four hundreds cells/well were seeded, and 8 wells were used for each clone. The cells were incubated for 7 hours and then fixed using 3% PFA. Data was collected using the autofocus screening microscope.

MDA-MB-231 cDNA migration screen: A cDNA library (1-3 kb fragments) of MDA-MB-231, highly migrating and metastatic breast carcinoma cell line, was generated and packed into pEYK retroviral vector by the group of Prof. J. Brugge (Harvard Medical School, USA). MCF-7, (a poorly metastatic, mammary tumor cell line), were infected with this cDNA library and screened for their migratory activity. In order to reduce the biohazardous danger MCF7-ER cells were used. These cells expresse ecotropic receptors on their surface and enable an infection procedure with ecotropic viruses that don't infect human. By calibrating the system for 30% rate infection 50,000 MCF7 cells were infected by the retroviral plasmid (pEYK3.1) expressing genes from the MDA-MB-231 cells. 48 hours following infection half of the infected cells were frozen and the rest of them were distributed as 20 cells/well in twenty-one plates of 96 wells plates; Since the infection rate is 30%, this means statistically that 1-6 different clones may be present per well. The cells were left to grow and reach confluence, and subsequently resuspended. Approximately 200 cells from each well were reseeded in 96 wells plate which had been pretreated with the beads. Cells were incubated at 37° C. for 7 hours, then fixed with 3% paraformaldehyde. Half of the cells from the original plate were maintained in culture and the other half were frozen in 96 well-V-shape bottom plates.

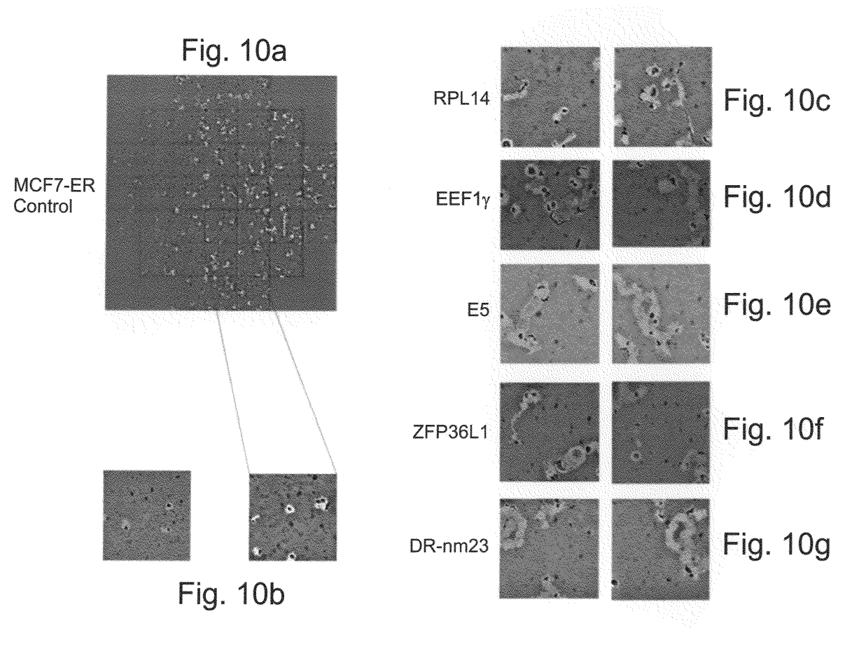

The phagokinetic tracks of the cells that were seeded on beads were recorded using the autofocus screening system. By looking at the 96 montages of the whole plate, wells could be identified that contain MCF7 cells, the majority of which don't migrate and have no long tracks, but that also comprise one or more cells with increased migration velocity (long track) within the same well. These wells were categorized as a candidate well containing 1-5 different genes. Only one of these genes is the migration-promoting gene in this specific well (FIG. 10). In the control cells that were GFP-MCF7 cells no long tracks were observed (FIG. 11). In order to identify the migration-inducing gene in the candidate well, the back-up plates that were maintained in culture were used. Cells were transferred from each parallel well of the candidate wells from the screen to 10 cm dish and put under selection conditions (zeocin 100 μg/ml). Once the cells were fully selected and reached a confluent state, the genomic DNA was purified from the cells, and digested by NotI restriction enzyme in order to rescue the entire pEYK 3.1 plasmid, which included the insert, from the genomic DNA. This was followed by self ligation of the plasmid, transformation of the ligation into competent cells and then plating of the bacteria on LB. Twenty colonies from each transformation reaction were taken and DNA mini-preps were prepared from it. Each and every one of the 20 DNAs went through digestion with the restriction enzyme AscI in order to rescue the insert. The aim here was to run all the 20 cut DNAs on Agarose gel in order to identify size differences, thus indicating the number of different genes which exist in the candidate well. One from each size-group was taken for sequencing. Fifteen candidates genes were rescued from the 6 candidate wells. All these genes were reintroduced to the MCF7 cells and the PKT assay were performed again to find “the one” in each well that had increased motility capacity to the cells.

Results

Identification of promigratory genes using the BC1000 cDNA migration screen: To identify genes that induce migration, MCF7 cells (non-migratory breast epithelial cells) were selected because they are stationary in the absence of exogenous mitogenic factors. For this screen, MCF7 cells were infected with retroviral vectors encoding genes known to be involved in breast cancer progression, but their role in cell migration is still not clear. 55 genes out of the BC1000 library were randomly selected. As control GFP-MCF7 cells were generated.

Each one of the 55 and the control were tested for their migration properties using the PKT system. For each clone 8 wells from the 96 well-plate were used. The PKTs were recorded using the auto-focus screening microscope system.

Nine promigratory candidates from this migration screen were identified (FIG. 7A-T). The candidates were as follows:

### 1. BIRC5 [GI: 21707886]

2. Milk fat globule EGF factor 8 protein (MFGE8) [GI: 13177647]
3. PKC zeta [GI: 14165514]
4. Rho GDI alpha (ARHGDIA) [GI: 20149550]

### 5. ErbB3 [GI: 12803738]

### 6. HOX B7 [GI: 15929846]

### 7. Chemokine CXL-ligand 6 (SCYB6) [GI: 15489286]

8. Calcium binding protein (CHP) [GI: 6005730]

### 9. FGF7 [GI: 15147244]

Each clone exhibited higher PKT area than the GFP-control. The cells populations vary in their migration capacity within the clones. Average value of each parameter for each candidate (as illustrated in Table 6 hereinbelow), indicate a statistically significant increase in the migration activity, as well as for most of the measured parameters.

TABLE 6

| | Area | Perimeter | Major axis | Minor axis | Axial |
|---|---|---|---|---|---|
| Gene name | (μm²) | (μm) | (μm) | (μm) | ratio |
| GFP | 4300 ± 2000 | 345 ± 105 | 100 ± 30 | 70 ± 20 | 1.5 ± 0.5 |
| n = 230 | | | | | |
| FGF7 | 5800 ± 4000 | 360 ± 160 | 125 ± 60 | 72 ± 20 | 1.7 ± 0.7 |
| n = 189 | p = 5.2 × 10⁻⁴ | N.S. | p = 4 × 10⁻⁴ | N.S. | p = 0.04 |
| HOXB7 | 5600 ± 3600 | 380 ± 110 | 120 ± 40 | 75 ± 20 | 1.6 ± 0.5 |
| n = 293 | p = 4.8 × 10⁻⁵ | p = 0.002 | p = 5 × 10⁻⁵ | p = 0.004 | p = 0.006 |
| PKC□ | 7500 ± 4400 | 410 ± 140 | 145 ± 55 | 80 ± 20 | 1.8 ± 0.6 |
| n = 303 | p = 3.4 × 10⁻²⁰ | p = 7 × 10⁻⁷ | p = 4.4 × 10⁻²⁰ | p = 3.9 × 10⁻⁶ | p = 3.8 × 10⁻⁶ |
| ERBB3 | 5700 ± 2900 | 370 ± 100 | 120 ± 35 | 75 ± 15 | 1.7 ± 0.6 |
| n = 261 | p = 2.1 × 10⁻¹⁷ | p = 0.01 | p = 4 × 10⁻⁹ | p = 0.04 | p = 1 × 10⁻⁴ |

| | Migration | Effective | | |
|---|---|---|---|---|
| | Velocity | Velocity | | |
| Gene name | (μm/h) | (μm/h) | Solidity | Roughness |
| GFP | 9 ± 4 | 9 ± 4 | 0.83 ± 0.1 | 2.3 ± 0.7 |
| n = 230 | | | | |
| FGF7 | 12 ± 9 | 11 ± 8 | 0.9 ± 0.1 | 1.9 ± 0.7 |
| n = 189 | p = 2 × 10⁻⁵ | p = 1.7 × 10⁻⁴ | p = 9.7 × 10⁻¹¹ | p = 6 × 10⁻³⁶ |
| HOXB7 | 11 ± 5 | 11 + 5 | 0.8 ± 0.1 | 2.3 ± 0.7 |
| n = 293 | p = 7.8 × 10⁻⁵ | p = 2 × 10⁻⁴ | N.S. | p = 1.4 × 10⁻⁹ |
| PKC□ | 16 ± 10 | 14 ± 7 | 0.9 ± 0.2 | 1.9 ± 0.5 |
| n = 303 | p = 8.2 × 10⁻²⁴ | p = 8.1 × 10⁻¹¹⁶ | p = 6 × 10⁻⁸ | p = 3.4 × 10⁻⁴² |
| ERBB3 | 12 ± 11 | 11 ± 8 | 0.85 ± 0.08 | 2.0 ± 0.6 |
| n = 261 | p = 4.6 × 10⁻⁸ | p = 1 × 10⁻⁶ | p = 0.006 | p = 8.7 × 10⁻²⁵ |

NS = not significant

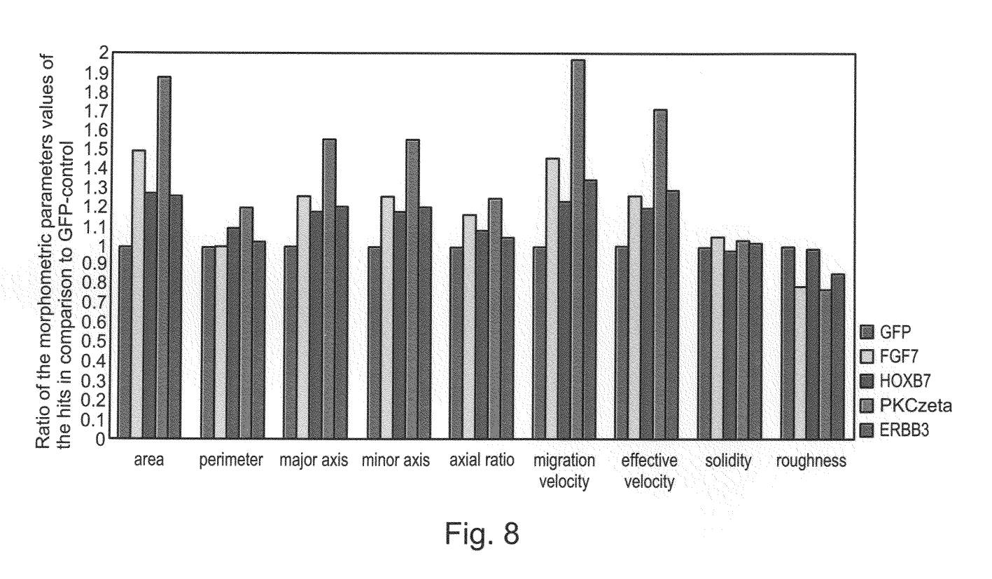

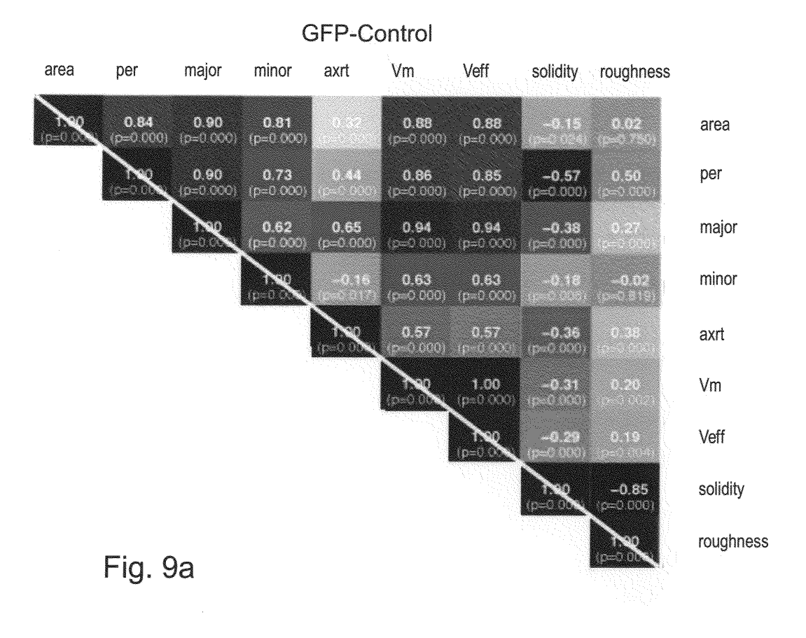

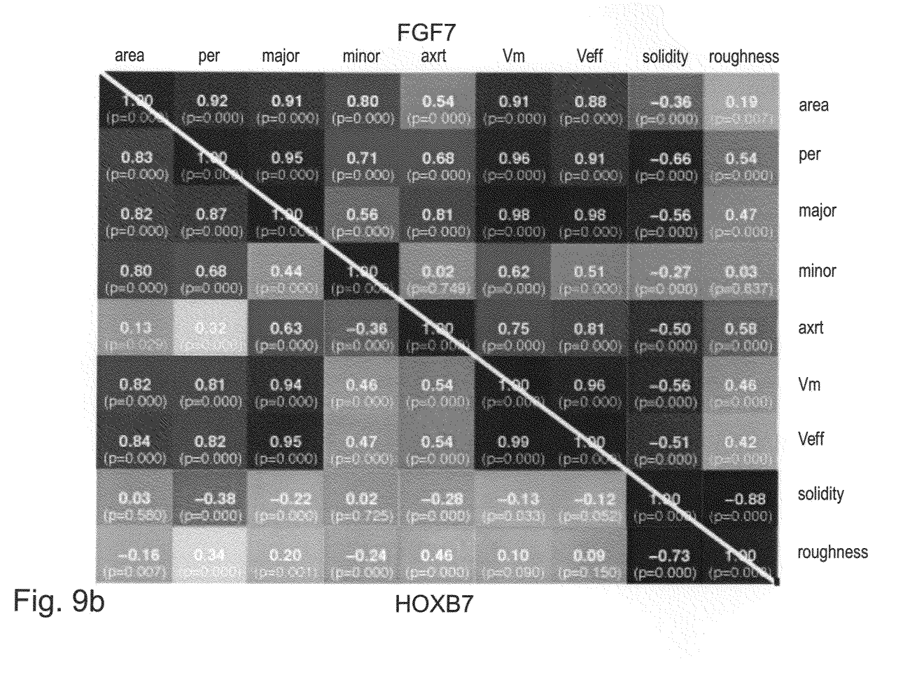

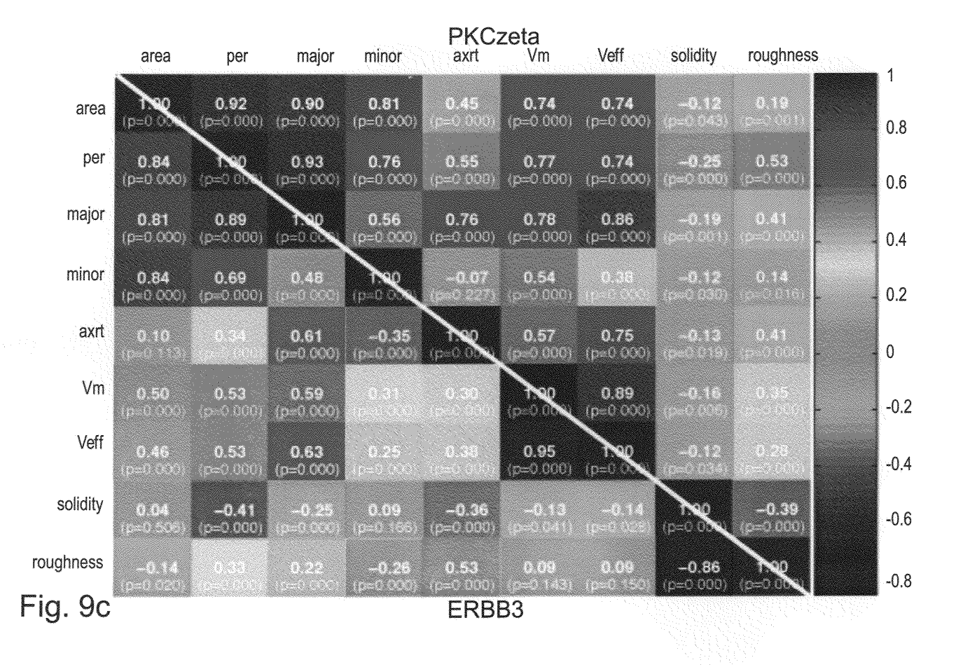

A more clear view of the results may be observed by looking at the 80^{th} percentile value of each parameter, indicating on the value that 20 percent of the tested population is above it. Ratio between 80^{th} percentile values of the candidate and the GFP-control is presented in FIG. 8. Analyzing the migratory behavior of each candidate from these results indicate on different migratory features. On one hand, FGF7 and high persistence migration with low lateral lamellar activity. Moreover, the migration velocity of PKCζ was almost two times greater than the control, FGF7 having a 50% increase in it's migration velocity. On the other hand, HOXB7 and ERBB3 clones comprised an approximate 30% increase in cellular migration velocity, the track border solidity value and roughness indicating a high lateral lamellar activity. In order to understand the impact of the changes of the parameters on each other, a correlation test was performed (FIG. 9A-C). The track's area of GFP-control cells showed a high positive and linear correlation with all the morphometric parameters but it was not correlated with the solidity, roughness and minor axis parameters, indicating high random activity of the lateral cell lamella. HOXB7 and ERBB3 clones also showed a poor correlation of the solidity and roughness parameters, but interestingly, minor axis was anti-correlated with axial ratio while minor axis and major axis was highly positively correlated. This indicates a persistence in migration, but with high lateral lamellar activity. PKC; and FGF7 clones exhibited linear anti-correlation between solidity and roughness and the rest of the morphometric parameters. This anti-correlation, especially with the track area and track perimeter indicates a smooth track border, which further indicates a high lamellar persistence. The linear positive correlation between major axis and axial ratio on the one hand, and the absence of any correlation between minor axis and axial ratio on the other hand, indicates a constant width and elongated shape of the tracks produced by these clones. This indicates persistence migration with a very low tendency to change migration direction within the migration process.

Identification of promigratory genes using the MDA-MB-231 cDNA migration screen: Twenty percent of this library were screened and five migration inducing genes were identified (FIGS. 10A-G). The genes that were identified were as follows:

1. Ribosomal protein L14E family (RPL14E) [GI:20810535]
2. Eukaryotic elongation factor gamma (EEF1gamma) [GI: 40226406]
3. Zinc finger protein 36 (C3Htype-like 1, ZFL36 μl) [GI: 15812179]
4. Nucleotide diphosphate kinase (DR-nm23) [GI: 12652978]
5. E5=Homo Sapiens chromosome 1 clone RP4-7 [GI: 22038620]

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-11. (canceled)

12. A method of treating a medical condition associated with cell migration, the method comprising administering to a subject in need thereof a therapeutically effective amount of an agent capable of modulating the activity or expression of ZFL36L1, thereby treating the medical condition associated with cell migration.

13. The method of claim 12, wherein said modulating is upregulating the activity or expression of ZFL36L1 and whereas the medical condition comprises a tissue damage.

14. The method of claim 13, wherein said tissue damage is selected from the group consisting of a wound, a spinal cord injury, brain injury, brain trauma and a neuronal disease or disorder.

15. The method of claim 12, wherein said modulating is downregulating the activity or expression of ZFL36L1 and whereas the medical condition is selected from the group consisting of cancer and cancer metastasis.

16. The method of claim 13, wherein said agent is an isolated polynucleotide which comprises a nucleic acid sequence encoding a ZFL36L1 polypeptide.

17. The method of claim 15, wherein said cancer is beast cancer.

18-114. (canceled)

115. A method of modulating cell migration, the method comprising contacting the cell with an agent capable of modulating the activity or expression of ZFL36L1, thereby modulating migration of the cell.

116. A method of treating a medical condition associated with cell migration, the method comprising administering to a subject in need thereof a therapeutically effective amount of an agent capable of modulating the activity or expression of PKC-ZETA, thereby treating the medical condition associated with cell migration.

117. The method of claim 116, wherein said modulating is upregulating the activity or expression of PKC-ZETA and whereas the medical condition comprises a tissue damage.

118. The method of claim 117, wherein said tissue damage is selected from the group consisting of a wound, a spinal cord injury, brain injury, brain trauma and a neuronal disease or disorder.

119. The method of claim 116, wherein said modulating is downregulating the activity or expression of PKC-ZETA and whereas the medical condition is selected from the group consisting of cancer and cancer metastasis.

120. The method of claim 117, wherein said agent is an isolated polynucleotide which comprises a nucleic acid sequence encoding a PKC-ZETA polypeptide.

121. The method of claim 119, wherein cancer is beast cancer.

122. A method of modulating cell migration, the method comprising contacting the cell with an agent capable of modulating the activity or expression of PKC-ZETA, thereby modulating migration of the cell.
